(19) United States
(12) Patent Application Publication
(10) Pub. No.: US 2026/0351365 A1
(43) Pub. Date: Nov. 5, 2026

(54) MULTI-MODEL VALIDATION AND LABEL-DEFERRED ASSEMBLY OF MACHINE-GENERATED ASSESSMENT OBJECTS

(71) Applicant: Lukasz R. Kiljanek, Gillette, WY (US)

(72) Inventor: Lukasz R. Kiljanek, Gillette, WY (US)

(21) Appl. No.: 19/668,770

(22) Filed: May 5, 2026

Related U.S. Application Data
(60) Provisional application No. 63/799,760, filed on May 5, 2025.
(60) Provisional application No. 63/854,410, filed on Jul. 30, 2025.
(60) Provisional application No. 63/869,058, filed on Aug. 22, 2025.

(51) Int. Cl. G16H 10/60; G06N 20/00

(52) U.S. Cl. CPC G16H 10/60; G06N 20/00

(57) ABSTRACT

Systems and methods are disclosed for multi-model assessment generation and validation. A system aggregates performance data and assessment parameters to generate an enriched prompt. A generation model processes the enriched prompt to generate a structured assessment object including an assessment content portion and response-option objects that include a correct response-option object and at least one incorrect response-option object. The response-option objects can be generated without user-facing labels, and the incorrect response-option object can be generated using the correct response-option object as context. Independent validator models audit the structured assessment object and return approval or rejection statuses without directly modifying the structured assessment object. In response to a rejection, the system regenerates at least a portion of the structured assessment object. In response to approval, the system assigns response labels, generates explanations, tracks references, presents an interactive assessment interface, and generates a score based on received inputs.

### CROSS-REFERENCE TO RELATED APPLICATIONS

The present application claims the priority benefit of U.S. provisional application No. 63/799,760 filed May 5, 2025 and entitled “AI Educational System,” U.S. provisional application No. 63/854,410 filed Jul. 30, 2025 and entitled “AI medical question generation,” and U.S. provisional application No. 63/869,058 filed Aug. 22, 2025 and entitled “Medical knowledge generator and AI blender,” the disclosures of which are hereby incorporated by reference in their entireties.

### TECHNICAL FIELD

This disclosure relates to training and using a suite of machine learning models to generate and validate assessment objects or other content, and more particularly, for systems using machine learning models to generate sets of questions with corresponding responses (e.g., both a correct response and incorrect response options that are plausible), validate technical and language accuracy, generate forensic audit trails, generate explanations corresponding to both correct and incorrect answers, track performance data, and update the machine learning models accordingly.

### BACKGROUND

Data platforms can include enormous volumes of data that can be difficult to parse through. Data platforms can be used to store sales data, health data, transaction data, location data, vehicle data, and/or other types of data. In some cases, data platforms can receive data continuously, and at a faster rate than any human could process such data.

Artificial intelligence (AI) refers to a class of computer algorithms that can process data and make intelligent decisions, for instance based on rules, heuristics, and/or prior learning. Machine learning (ML) models are a subset of AI algorithms. ML models learn how to process input datasets to generate desired output datasets based on training data, which can for instance include examples of appropriate output datasets for different possible input datasets. In some cases, ML models can continue to learn over time. Neural networks (NNs) are a type of ML model with interconnected layers of nodes (referred to as “neurons”) that are used to process information.

Generating high-quality assessment materials with artificial intelligence can be technically difficult because the generated questions should be accurate, appropriately varied, and suited to a learner's level while avoiding plausible but incorrect or misleading content. These challenges can be especially significant in fields such as medicine, where small factual errors, poorly calibrated answer choices, or repetitive question patterns can reduce the usefulness of the assessment materials.

### SUMMARY

Systems and methods are disclosed for multi-model assessment generation and validation. A system aggregates performance data and assessment parameters to generate an enriched prompt. A generation model processes the enriched prompt to generate a structured assessment object including an assessment content portion and response-option objects that include a correct response-option object and at least one incorrect response-option object. The response-option objects can be generated without user-facing labels, and the incorrect response-option object can be generated using the correct response-option object as context. Independent validator models audit the structured assessment object and return approval or rejection statuses without directly modifying the structured assessment object. In response to a rejection, the system regenerates at least a portion of the structured assessment object. In response to approval, the system assigns response labels, generates explanations, tracks references, presents an interactive assessment interface, and generates a score based on received inputs.

In an example, a method is provided for multi-model assessment generation and validation. The method includes aggregating assessment parameters to generate an enriched prompt; processing the enriched prompt with a generation model to generate a structured assessment object that includes an assessment content portion and a plurality of response-option objects, wherein the plurality of response-option objects include a correct response-option object and at least one incorrect response-option object; auditing the structured assessment object with a plurality of independent validator models; regenerating at least a portion of the structured assessment object based on receipt of a rejection from at least one of the plurality of independent validator models to generate an updated structured assessment object; and generating a score based on a plurality of inputs received through an interactive assessment interface, wherein the interactive assessment interface outputs the structured assessment object, and wherein one of the plurality of inputs selects at least one of the plurality of response-option objects. In another example, a system is provided for multi-model assessment generation and validation. The system includes a memory storing instructions and a processor that executes the instructions. Execution of the instructions by the processor causes the processor to perform operations comprising: assessment parameters to generate an enriched prompt; processing the enriched prompt with a generation model to generate a structured assessment object that includes an assessment content portion and a plurality of response-option objects, wherein the plurality of response-option objects include a correct response-option object and at least one incorrect response-option object; auditing the structured assessment object with a plurality of independent validator models; regenerating at least a portion of the structured assessment object based on receipt of a rejection from at least one of the plurality of independent validator models to generate an updated structured assessment object; and generating a score based on a plurality of inputs received through an interactive assessment interface, wherein the interactive assessment interface outputs the structured assessment object, and wherein one of the plurality of inputs selects at least one of the plurality of response-option objects.

In another example, a non-transitory computer-readable storage medium is provided, having embodied thereon a program. The program is executable by a processor to perform a method of multi-model assessment generation and validation. The method includes aggregating assessment parameters to generate an enriched prompt; processing the enriched prompt with a generation model to generate a structured assessment object that includes an assessment content portion and a plurality of response-option objects, wherein the plurality of response-option objects include a correct response-option object and at least one incorrect response-option object; auditing the structured assessment object with a plurality of independent validator models; regenerating at least a portion of the structured assessment object based on receipt of a rejection from at least one of the plurality of independent validator models to generate an updated structured assessment object; and generating a score based on a plurality of inputs received through an interactive assessment interface, wherein the interactive assessment interface outputs the structured assessment object, and wherein one of the plurality of inputs selects at least one of the plurality of response-option objects.

In another example, a system is provided for multi-model assessment generation and validation. The system includes means for aggregating assessment parameters to generate an enriched prompt; means for processing the enriched prompt with a generation model to generate a structured assessment object that includes an assessment content portion and a plurality of response-option objects, wherein the plurality of response-option objects include a correct response-option object and at least one incorrect response-option object; means for auditing the structured assessment object with a plurality of independent validator models; means for regenerating at least a portion of the structured assessment object based on receipt of a rejection from at least one of the plurality of independent validator models to generate an updated structured assessment object; and means for generating a score based on a plurality of inputs received through an interactive assessment interface, wherein the interactive assessment interface outputs the structured assessment object, and wherein one of the plurality of inputs selects at least one of the plurality of response-option objects.

This summary is not intended to identify important features of the claimed subject matter, nor is it intended to be used in isolation to determine the scope of the claimed subject matter.

### BRIEF DESCRIPTION OF THE DRAWINGS

Illustrative embodiments of the present application are described in detail below with reference to the following figures:

FIG. 1 is a block diagram illustrating a system for generating, validating, updating, and tracking assessment objects, in accordance with some examples

FIG. 2 is a block diagram illustrating an architecture of a system for machine-learning-model-based assessment generation and interfacing, in accordance with some examples;

FIG. 3 is a block diagram illustrating an example of an artificial intelligence (AI) system for training, use of, and/or updating of one or more machine learning (ML) model(s) that are used to generate an enriched prompt, a structured object, validation(s), explanation(s), score(s), retrieval-augmented generation (RAG) query(s), and/or tool command(s), in accordance with some examples;

FIG. 4 is a conceptual diagram illustrating interactive user interfaces (UIs) for presenting assessment objects and receiving inputs selecting response-option objects. The user interfaces include a UI 410 that includes a question and multiple response options, a UI 420 in which an incorrect response option was selected, and a UI 430 in which a correct response option was selected.

FIG. 5 is a flow diagram illustrating a process for multi-model assessment generation and validation, in accordance with some examples; and

FIG. 6 is a block diagram of an exemplary computing device that may be used to implement some aspects of the technology.

### DETAILED DESCRIPTION

The following description provides examples of systems, methods, computer-readable media, and user interfaces for generating, validating, updating, and tracking assessment objects and other generated content objects. Technical generation environments can require a computing system to combine heterogeneous input data, including prompts, performance records, clinical parameters, references, media, sensor data, source-material records, and feedback, while producing output objects that can be rendered by an interface, scored, audited, reprocessed, selected for a user, and stored. The inputs can differ in data structure, timing, provenance, authorization level, and output-format requirements. These conditions create technical problems related to schema validity, context freshness, duplicate model requests, citation provenance, numerical verification, media verification, and traceability between an output object and the inputs, models, tools, and validations that produced the output object. Additional technical challenges can include context-window saturation, inconsistent field naming across model outputs, unavailable or stale references, model-output race conditions, and difficulty preserving a stable mapping between generated content and the data used to generate the content. For example, a first model output can identify a reference passage while a later validator output can identify a conflicting passage, and the system can preserve both machine-readable records without allowing either output to overwrite the other until a controlled update step is reached.

The technical problems can be amplified when assessment content is generated by asynchronous local and remote model calls, retrieval operations, tool calls, and verification routines. For example, a canceled or superseded generation session can leave a stale model output available to a downstream renderer, a repeated request can consume accelerator or network resources to produce a duplicate response, and a validator output that is permitted to modify a generated object directly can obscure which model or tool changed a field. In addition, a response label assigned before validation can leak answer-position information into generated explanations, examples, screenshots, or stored question-bank records.

The examples described herein address these technical problems by configuring a computer pipeline that enriches an input prompt, generates a structured assessment object, validates the structured assessment object using independent validator models and deterministic verification logic, and routes rejection data back to a generation model through a controlled feedback path. The pipeline can defer user-facing response-label assignment until after approval, store provenance and validation metadata in a reference tracker, maintain generation-session identifiers and cancellation state, and use request hashes, response hashes, annotation identifiers, model-registry records, and tool-interface records to coordinate model and tool activity.

By representing assessment content, response-option objects, references, explanations, scores, and validation results as machine-readable fields rather than only as unstructured text, the configured system can validate object fields independently, preserve mappings among fields, and regenerate only a rejected portion of an object when appropriate. The structured representation also permits an interactive interface to render a question, a clinical vignette, response-option objects, explanations, media, and scores while the backend maintains an audit trail of the model identities, tool commands, validation statuses, references, and user interactions associated with the displayed content. The machine-readable fields can include field names, field types, schema versions, lock states, validation states, citation pointers, confidence values, and regeneration scopes. These fields can allow the system to regenerate a distractor object, explanation object, citation object, or media object without reprocessing an approved question object or approved response-option object.

The configured system can improve operation of the computing environment by reducing invalid output retries, reducing duplicate network and model calls, preventing stale outputs from being presented after cancellation or supersession, and limiting validator components to comment-only permissions so that state-changing operations are performed through a controlled regeneration step. Local sentence-aware reference verification, deterministic numerical checks, model-provider selection based on declared capabilities, and cancellation-aware scheduling can reduce latency, memory consumption, bandwidth consumption, and accelerator utilization while improving reproducibility, provenance tracking, and interface reliability. In batch-processing examples, the system 100 can process grouped assessment objects or explanation sets with shared definitions, shared prompt instructions, reusable annotations, deterministic length measurements, per-item approval ledgers, and shrinking active working sets, thereby reducing repeated prompt tokens and avoiding disturbance of approved content while other items in the batch are regenerated or revalidated.

An assessment object can include any computer-readable object that represents an assessment item, such as a medical question, a non-medical question, a simulation prompt, a case vignette, a response set, one or more explanations, one or more citations, one or more metadata records, one or more scores, or combinations thereof. More generally, a content object generated or validated by the system can include educational content, training content, reference content, media content, a document, a game, an image, an audio item, a video item, a flashcard, a poll, a simulation object, or another generated output. A structured assessment object or content object can be represented using JavaScript Object Notation (JSON), eXtensible Markup Language (XML), YAML Ain't Markup Language (YAML), a database record, a protocol buffer, a table, or another structured format. Although many examples below refer to medical education, clinical parameters, and clinical vignettes, the same techniques can be used for other assessment or content-generation domains, such as science, engineering, compliance, language training, professional certification, safety training, operational training, customer support, software training, and other knowledge or skill assessments. Similarly, references to a student can include a learner, trainee, clinician, operator, user, employee, candidate, or other person or account for whom an assessment object or content object is generated, presented, scored, selected, or tracked. Terms such as assessment object, assessment item, generated item, item object, question object, content object, prompt-response object, evaluation object, and learning object can be used to refer to a computer-readable object that is generated, validated, presented, scored, stored, or revalidated by the system. Narrower examples of an assessment object include a multiple-choice medical question, a clinical vignette with answer candidates, a flashcard record, a true-or-false prompt, a case-simulation prompt, a spoken quiz item, and an interactive interface state.

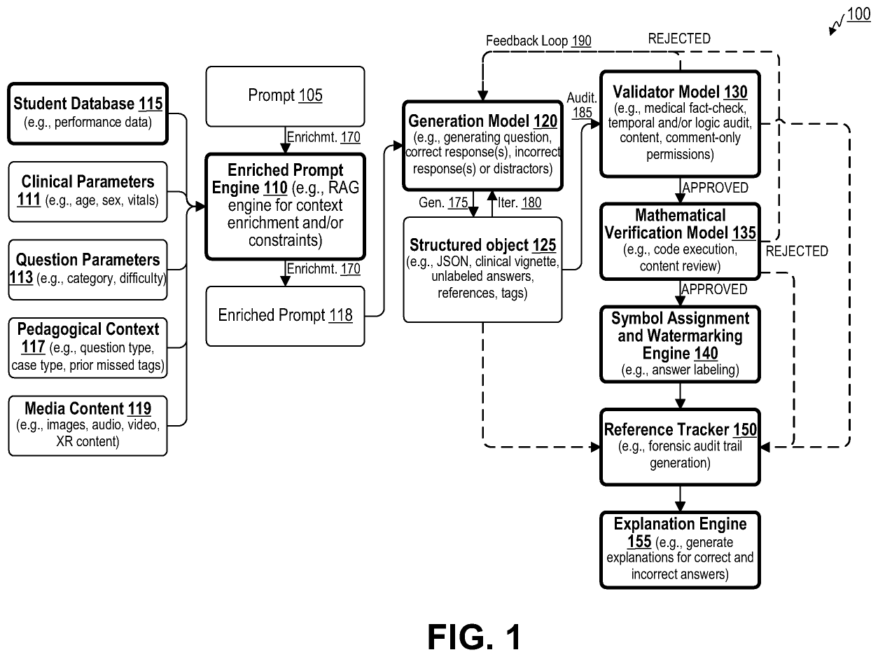

FIG. 1 is a block diagram illustrating a system 100 for generating, validating, updating, and tracking assessment objects. The system 100 can receive a prompt 105, such as an original user prompt, administrator prompt, batch-generation prompt, or application programming interface (API) request. The prompt 105 can identify a topic, domain, assessment objective, desired difficulty, target user, output format, or other generation criteria. In some examples, the prompt 105 is not used directly by the generation model 120, but is enriched, normalized, constrained, or otherwise transformed before generation.

The system 100 includes an enriched prompt engine 110. The enriched prompt engine 110 can aggregate and process clinical parameters 111, question parameters 113, data from a student database 115, pedagogical context 117, media content 119, and/or other contextual data to generate an enriched prompt 118. The clinical parameters 111 can include patient age, sex, vital signs, laboratory values, imaging findings, medical history, medication history, social history, allergies, or other clinical variables. The question parameters 113 can include category, subcategory, topic, difficulty, question type, case type, target examination, target audience, language, format, or other assessment parameters. The student database 115 can store performance data, prior answers, tags of missed questions, percentile ranks, frequency of use, feedback history, and other learner-specific or cohort-specific data. The student database 115 can be referred to as a learner database. The pedagogical context 117 can include learning objectives, case-type definitions, question-type definitions, difficulty rules, number of relevant and irrelevant findings, similarity requirements for distractors, and prior missed tags. The media content 119 can include images, audio, video, extended reality (XR) content, three-dimensional content, documents, or other media used as input to, or generated as output from, the system 100.

The enriched prompt engine 110 can perform enrichment 170 by combining the prompt 105 with one or more of the clinical parameters 111, question parameters 113, student database 115, pedagogical context 117, and media content 119. Enrichment 170 can include retrieval-augmented generation, query execution, template filling, prompt normalization, difficulty calibration, insertion of constraints, translation, redaction, context-window management, or selection of one or more model-specific prompt formats. The output of enrichment 170 can be the enriched prompt 118. Context-window management can include token budgeting, chunk selection, retrieval filtering, context compression, source-priority ranking, omission of locked approved content, and splitting a prompt into role-specific prompt segments. For example, the enriched prompt engine 110 can provide a long clinical context to a generation model while providing a shorter reference-verification context to a validator model, thereby reducing unnecessary token transfer and accelerator memory usage.

The generation model 120 can process the enriched prompt 118 during generation 175 to generate a structured object 125. The structured object 125 can include a structured assessment object, such as a JSON object having an assessment content portion, a question stem, a clinical vignette, a correct response-option object, one or more incorrect response-option objects, one or more references, one or more tags, one or more numerical values, and/or metadata. In some examples, the generation model 120 generates the correct response-option object and the incorrect response-option objects in a single model pass. In other examples, the generation model 120 performs iteration 180 by generating response-option objects in multiple passes, such as by generating a correct response-option object and then generating one or more distractor response-option objects using the correct response-option object and/or previously generated distractor response-option objects as context. The structured object 125 can store internally labeled fields, such as a correct-answer field and one or more incorrect-answer fields, while user-facing labels and presentation order are assigned later by the symbol assignment and watermarking engine 140 during quizzing, session assembly, or interface presentation. The later label assignment can be randomized per user, per session, per assessment object, or per distribution event, and can be used for answer-order normalization, watermarking, fingerprinting, and tracing unauthorized dissemination. The structured object 125 can be referred to as a structured assessment object, as an assessment object, as a question-response pair, as a question-response set, as a question and its responses, or a combination thereof.

In some examples, the correct response-option object, incorrect response-option objects, or both are generated serially, generated in parallel, generated by a shared generation model, generated by different generation models, or generated using a combination of serial and parallel model calls. Likewise, multiple structured assessment objects can be generated one after another or as a parallel group using the same model, different models, or a model ensemble selected by the model registry. The generation model 120 can also be referred to as a generator, item generator, content-generation model, assessment-generation model, response-generation model, prompt-response model, or structured-output model. The response-option objects can also be referred to as candidate answer objects, answer-option objects, selectable option objects, distractor objects, foil objects, decoy answer objects, or response candidates, depending on the level of breadth desired.

The question parameters 113 and pedagogical context 117 can include a structured context dictionary for a generation session. The structured context dictionary can include a category path, topic, question type, case type, difficulty level, teaching point, patient context, vital signs, diagnostic results, social history, allergy history, prior response records, missed-question tags, cohort percentile data, and custom prompt constraints. The structured context dictionary can also include definitions of the question type, case type, and difficulty level so that the generation model 120 and validator model 130 receive the same machine-readable interpretation of those parameters. The structured context dictionary can also be referred to as a context object, prompt-state record, generation context record, session context, parameter bundle, or assessment-generation state.

Difficulty rules in the pedagogical context 117 can specify a number of implicit reasoning steps between the assessment content portion and the correct response-option object, a number of relevant findings, a number of irrelevant but plausible findings, a target similarity between the correct response-option object and distractor response-option objects, and a target length similarity among response-option objects. For example, an easier difficulty level can specify fewer implicit reasoning steps, fewer data points, fewer irrelevant findings, and more visibly different distractors, while an expert difficulty level can specify multiple implicit reasoning steps, more data points, more past-history items, and distractors that differ from the correct response-option object by small numerical, wording, or conceptual differences. The system 100 can also apply quality metrics to response-option objects, including uniformity of answer-choice length, semantic distance among answer choices, conceptual distance among answer choices, word-overlap measurements, numerical-value separation, reference vintage, reference-source quality, and whether the correct response-option object is disproportionately long or short. For example, deterministic code can measure answer-choice lengths across a batch and reject, flag, or regenerate one or more assessment objects when more than a threshold percentage of the batch has the correct response-option object as the longest response-option object.

The enriched prompt engine 110 can also insert technical and clinical guardrails into the enriched prompt 118. The guardrails can instruct the generation model 120 to avoid answer-revealing language, avoid grouping findings by relevance, avoid embedding response-option objects in the question stem, maintain chronological consistency in a clinical history, maintain compatibility between a diagnostic test and the type of result reported for that test, and avoid unnecessary invasive or expensive testing unless a generated scenario provides a reason for such testing. These guardrails provide machine-readable constraints that reduce invalid model outputs and reduce repeated regeneration cycles.

The enriched prompt engine 110, reference tracker 150, validator model 130, or another subsystem can also enforce reference-source constraints, which can also be referred to as filters or constraints. The reference-source constraints can specify publication-date windows, such as references from the last five years or another recency window; geographic or organizational source rules, such as United States guidelines, United Kingdom guidelines, European guidelines, or professional-society guidelines; source-type rules, such as peer-reviewed journals, open-access sources, non-paywalled sources, clinical guidelines, or government sources; and exclusion rules, such as excluding blogs, textbooks, low-quality sources, unsupported summaries, or sources exceeding a length threshold. The reference-source constraints can be used during generation, validation, explanation generation, explanation validation, and revalidation. For example, the reference-source constraints can require United States medical journals published within a first time window, such as five years, for one category, and can require United Kingdom endocrinology journals published within a second time window, such as ten years, for another category. The constraints can also specify that pre-publication manuscripts, pre-peer-review materials, unpublished drafts, or sources that fail a journal-legitimacy check are excluded, down-weighted, flagged for review, or routed to an additional validator model. The reference-source constraints can also be referred to as evidence filters, source filters, citation policies, reference-quality rules, source-vetting criteria, or provenance rules.

The validator model 130 can represent one validator model, a plurality of validator models, or a validator model array. The validator model 130 can perform auditing 185 on the structured object 125. Auditing 185 can include content verification, medical fact-checking, mathematical verification, temporal verification, logical verification, language review, citation verification, formatting review, policy review, and/or other validation operations. In some examples, a first model is configured as the generation model 120 and two or more additional models are configured as validator models 130, although other model counts, model providers, model families, and model-role assignments can be used. In some examples, each validator model 130 can verify content, references, calculations, formatting, and explanation mapping, rather than being limited to a single validation category. In some examples, the validator model 130 is restricted to comment-only permissions with respect to the structured object 125. As a result, the validator model 130 can approve, reject, comment on, or score the structured object 125 without directly editing the structured object 125. A validator model 130 can also be referred to as a verifier, auditing model, critic model, scoring model, review model, adjudicator model, medical-accuracy model, citation-verification model, or quality-control model. Using a validator score rather than only free-form validator text can improve computer operation by allowing approval logic to be evaluated by deterministic comparison circuitry, database queries, or rules rather than by repeated natural-language interpretation.

A validator model 130 can also generate a validation score for the assessment content portion, the correct response-option object, one or more incorrect response-option objects, references, explanations, or combinations thereof. An approval or rejection can be based on comparison of an individual validation score, an average validation score, a minimum validation score, a maximum validation score, or a weighted validation score to one or more validation thresholds. In some examples, the score generated by the validator model 130 can be based in part on the quality, prestige, or reputation of the reference (e.g., the quality, prestige, or reputation of the scientific journal or publication), whether the reference is peer-reviewed or pre-peer-review, whether the reference is published or unpublished (e.g., an unpublished draft), and the like. In some examples, the system 100 can use multiple validator models (e.g., at least one validator model 130, at least one mathematical verification model 135, and the like). An approval or rejection can be based on consensus, or lack thereof, among at least a threshold number of models (e.g., at least N out of M validator models score the assessment object above or below a threshold, an average score of the scores generated by the validator models is above or below a threshold, a maximum score among the scores generated by the validator models is above or below a threshold, a minimum score among the scores generated by the validator models is above or below a threshold, or a combination thereof). In some examples, a separate validator model is omitted, and the generation model 120 performs self-validation, confidence scoring, reference checking, or calculation checking before the structured object 125 is accepted or passed to downstream components.

In some examples, the system 100 stores prompt-configuration records for multiple prompt families. Non-limiting examples of prompt families include a question-generation prompt, one or more question-validation prompts, an explanation-generation prompt, and one or more explanation-validation prompts. The prompt-configuration records can be stored in the data store system(s) 230 and/or data store(s) 380 and can be selected using the model registry. A prompt-configuration record can identify a model role, a model provider, a batch size, a target output schema, available tools, reference-source constraints, deterministic verification rules, length requirements, and feedback fields used to communicate with other model roles.

A question-generation prompt can instruct the generation model 120 to generate one or more assessment objects in a batch. The batch can include N multiple-choice questions or another number of content objects. The prompt can instruct the generation model 120 to use web-search or retrieval tools to verify references, guidelines, and domain facts, and to use code execution or another deterministic verification tool for calculations such as estimated glomerular filtration rate, body mass index, drug dose, anion gap, date interval, unit conversion, or another calculated value. The prompt can also specify that answer-choice lengths and stem lengths are measured by code after model output rather than estimated by the model, and can specify minimum lengths, maximum lengths, and distribution requirements for a batch. The one or more assessment objects in the batch can be generated serially or in parallel, and different questions, response-option objects, references, or explanations in the batch can be assigned to the same generation model 120 or to multiple generation models. When the assessment objects in the batch relate to a shared subject, such as a common medical topic, the prompt can reuse a shared set of retrieved references, definitions, or journal passages across multiple questions to reduce repeated retrieval and validation operations. This can improve efficiency of the system, reduce power usage by the system 100, reduce heat generation by the system 100, reduce need for heat dissipation components by the system 100, reduce wear and tear on the system 100, reduce bandwidth usage (e.g., from repeated retrieval of references) by the system 100, increase throughput of the system 100, or a combination thereof.

In some examples, the system 100 applies anti-leakage quality rules to response-option objects. For example, deterministic code can measure the length of each response-option object and determine whether the correct response-option object is the longest or shortest response-option object. If more than a threshold proportion of a batch, such as more than 20 percent of a batch, has the correct response-option object as the longest response-option object, shortest response-option object, or otherwise statistically distinctive response-option object, the system 100 can reject those assessment objects, reject the batch, regenerate affected response-option objects, or provide validator feedback to the generation model 120. Similar quality metrics can evaluate semantic distance, conceptual distance, word overlap, numerical-value distance, answer-choice parallelism, and whether distractors differ from the correct response-option object by clinically or technically meaningful differences. The metadata communication layer can be implemented as a sideband channel, inter-model message bus, audit channel, coordination record, review ledger, queue message, or non-user-facing metadata field. Separating the metadata communication layer from the assessment content can reduce interface-rendering errors by preventing validator notes, source requests, or inconsistency markers from being displayed as part of a learner-facing question.

In some examples, the system 100 maintains a metadata communication layer that is separate from the assessment content fields of the structured object 125. The metadata communication layer can include requests from a generating model to a validator model, responses from a validator model to a generating model, requests for assistance locating references, explanations of why a model selected a reference or response-option object, notices that a model cannot locate support for a fact, inconsistency reports, per-question feedback, and other model-to-model coordination data. The metadata communication layer can allow models to advise one another, identify unresolved issues, converge on a consensus, and preserve debugging information without causing such communications to appear as part of the question stem, response-option objects, explanation text, or other user-facing assessment content. For example, a validator model 130 can ask the generation model 120 to identify where a clinical fact was found, and the generation model 120 can respond with a journal name, passage identifier, uniform resource locator (URL), pointer, link, data reference, or confidence value, through the metadata communication layer. The metadata communication layer can also carry requests to resolve conflicting references, proposals for improved references, reviewer-style comments from one model to another, and syntax-marked issue reports, such as bracketed issue codes or asterisk-prefixed lines that identify inconsistent prompt context or requested user clarifications. The metadata communication layer can be referred to as an inter-model communication layer, protocol, and/or interface.

In some examples, a validation prompt instructs one or more validator models 130 to evaluate generated questions using measured length data, category definitions, question-type definitions, case-type definitions, difficulty rules, reference constraints, and deterministic verification results. The validator models can be instructed to return an approval or rejection flag for each assessment object, comment only on portions that require improvement, tag feedback to the correct assessment object or question number, identify prompt inconsistencies, and avoid suggesting changes to locked category, topic, question-type, case-type, or difficulty parameters unless those parameters are themselves identified as invalid inputs.

In some examples, an explanation-generation prompt instructs an explanation engine 155 or another generation model to generate explanation(s) 338 only after the associated assessment content has been approved. The explanation-generation prompt can instruct the model not to modify approved question text or response-option objects, to generate an overall explanation, a correct-response explanation, incorrect-response explanations, and a teaching pearl, and to begin each explanation with a verbatim anchor or other identifier corresponding to the response-option object being explained. In some examples, dose, route, timing, numerical values, units, and schedule information in an explanation must match the corresponding response-option object, and explanation length can be measured programmatically after model output. In some examples, the assessment content portion can be locked before response-option objects are generated or refined, or the response-option objects can be locked before the assessment content portion is finalized. References, annotations, and explanations can be locked before or after one another, and can be treated as part of the structured object 125 in some examples or as associated records linked to the structured object 125 in other examples.

In some examples, an explanation-validation prompt instructs one or more validator models 130 to verify that each explanation maps to the correct response-option object, repeats numerical and dosing information accurately, uses an appropriate depth and vocabulary for the difficulty level, and is supported by verified references. The validator models can return approval or rejection statuses for individual explanation sets. Explanation sets that are approved by a threshold number of validator models can be stored aside and omitted from later refinement prompts so that later validator or refiner calls operate on a smaller active working set while approved content remains unchanged.

The prompt-configuration records can also include a shared definitions dictionary. The shared definitions dictionary can define question types, case types, difficulty levels, categories, topics, allowed media types, and batch requirements once, and later prompt families can reference the definitions by identifier rather than repeating the full definitions. For example, question-type definitions can include most-likely diagnosis, best next diagnostic step, mechanism of action, management, underlying pathology, treatment approach, clinical features, epidemiology, diagnostic failure scenario, treatment failure scenario, complications, prevention strategies, common clinical pitfalls, common legal pitfalls, recent changes, clinical trials, natural history, emergency priorities, cost effectiveness, patient communication, differential diagnosis by physical examination and history, differential diagnosis by testing, differential diagnosis by any means, and contraindications and precautions.

Case-type definitions can include emergency, routine visit, typical presentation, complicated condition, patient in critical condition, tricky question with subtle findings, common clinical scenario, rare disease presentation, emergency-department case, multi-patient scenario, resource-limited setting, pregnant patient, or other case types. Difficulty definitions can include easy, moderate, hard, expert, or another difficulty level, and can specify target audience, target success rate, cognitive load, number of relevant findings, number of irrelevant findings, amount of patient context, closeness of distractors to a correct response-option object, and whether the generated assessment should test recall, application, integration, triage, expert judgment, or another skill. Other calculation examples include renal-dose adjustment, pediatric weight-based dosing, infusion-rate calculation, corrected sodium, corrected calcium, body surface area, risk-score calculation, date-interval calculation, and unit conversion between metric and non-metric units. Routing such calculations through deterministic verification logic can reduce reliance on probabilistic language-model arithmetic and can generate reproducible calculation records for later audit.

The mathematical verification model 135 can execute or invoke deterministic verification logic, such as code execution, symbolic computation, arithmetic verification, unit conversion, formula verification, or another verification program. For example, the mathematical verification model 135 can calculate an estimated glomerular filtration rate (eGFR), body mass index (BMI), dose, ratio, date interval, or other numerical value and compare the calculated value with a value included in the structured object 125. In some examples, mathematical verification is performed by a validator model 130, by the generation model 120, by a separate application service, by a deterministic calculator, or by another model or program that extracts values and context from the structured object 125 using natural language processing, an ML model, a rule-based parser, or combinations thereof. The numerical value can appear in the assessment content portion, the correct response-option object, an incorrect response-option object, an explanation, a reference annotation, or another field, and the mathematical verification model 135 can verify the value before or after another validator model 130 reviews the content. In an illustrative example, the mathematical verification model 135, if a structured object 125 (e.g., a question or response other portion of an assessment object) identifies a BMI of a patient (e.g., real or simulated patient), and includes the height and weight (or mass) of the patient, the mathematical verification model 135 can verify that the BMI is accurate by running code (e.g., as one of the tool(s) 385 via tool command(s) 355) to calculate the BMI as the weight (or mass) divided by the height squared, in some cases multiplied by a conversion factor such as 703 (if in imperial units of pounds and inches).

In some examples, the system 100 processes sensor data and biofeedback data as part of assessment generation, assessment selection, pacing, remediation, or learning-experience optimization. The sensor data can include electroencephalography data, pupillometry data, eye-tracking data, gaze data, camera data, audio data, speech data, vital-sign data, heart-rate data, blood-pressure data, oxygen-saturation data, skin-moisture data, temperature data, motion data, electromagnetic-signal recordings, implantable-sensor data, external-electrode data, user-entered mood data, medical-history data, language-preference data, age data, or other authorized input data. The system 100 can analyze such data using AI models, statistical models, or rule-based logic to select a next assessment object, generate a question set, repeat content, change a media modality, adjust difficulty, or update a study path.

In some examples, the system 100 can interface with output hardware that provides audio, visual, haptic, electrical, electromagnetic, or other stimulation under applicable permission and safety controls. Such output hardware can include speakers, displays, haptic actuators, stimulation interfaces, external electrodes, implantable leads, cortical leads, or other devices. The output hardware can be configured to provide study content, reinforcement signals, biofeedback signals, reward-associated signals, or memory-supporting signals during an awake state, sleep state, sedation state, review session, or another authorized operating state. In some examples, the system 100 can generate control data associated with brain regions or neural systems involved in memory, attention, reward, or learning, such as the hippocampus, cortex, or reward-related circuits, without requiring any particular biological effect in every implementation.

In some examples, the system 100 uses cloud processing, edge processing, or a hybrid architecture to model study paths for learners or other users. A study-path model can process prior responses, prior incorrect responses, score trends, confidence values, sensor signals, preferences, available time, desired examination outcome, target long-term retention, and teacher or administrator inputs. The study-path model can select newly generated assessment objects, retrieve cached assessment objects, generate a batch of assessment objects, or choose a sequence of questions for a learner based on AI-based or statistical analysis.

In some examples, the system 100 can translate assessment content, explanation content, and feedback content into one or more languages and can apply the same generation, validation, regeneration, and feedback-loop techniques to translated content. For example, a first model can translate a structured assessment object, one or more validator models can validate the translated object for accuracy and language quality, and a generation or refinement model can regenerate a portion of the translated object based on validator feedback. The translation can be performed by a language-specific model, a multilingual model, or a model selected for a target language, such as translating English assessment content into Polish assessment content, or vice versa. The system 100 can also translate between assessment formats, such as converting a multiple-choice question into a true-or-false item, flashcard, oral quiz, slide deck (e.g., .pptx format), word-processing document (e.g., .docx or .rtf format), spreadsheet (e.g., .csv or .xlsx format), a Portable Document Format (.pdf) document, a web-based online quiz or test format, an app-based quiz or test format running in a locally-installed application, or a combination thereof.

In some examples, the system 100 can generate and validate non-text content or derivative content. For example, the system 100 can generate images, infographics, audio files, video files, animations, three-dimensional scenes, extended-reality scenes, flashcards, polls, question-of-the-day content, question-of-the-week content, books, handouts, Portable Document Format (PDF) files, word-processing documents, slide decks, games, or other content based on one or more structured assessment objects. The system 100 can validate such content using one or more models configured for image, audio, video, text, code, or multimodal validation, and can provide model feedback to the generation model 120 to regenerate an image, audio clip, video clip, game, document, or other media object. For video or animation, the generated content can include text-to-speech audio and lip-movement animation synchronized to generated speech, and the validation can check timing, spoken content, subtitle text, and correspondence to the structured object 125. In one implementation, an audio file generated from approved assessment text can be transcribed by a speech-to-text tool and compared with the structured object 125 to detect omitted response options, changed labels, or mismatched explanations before the audio file is released.

In some examples, audio, video, image, sensor, or interaction data can trigger generation of educational material, assessment material, remediation material, or another content object. For example, the system 100 can process authorized recorded audio, video, images, or sensor data from a learning session, patient encounter, simulation, lecture, or review session and generate a corresponding assessment object, explanation, study note, flashcard, game, book section, or media item. The interaction data can include a doctor-patient interaction, doctor-learner (doctor-student) interaction, learner-teacher (student-teacher) interaction, simulation encounter, clinical debrief, or point-of-care recall event. Before the interaction data is used, in some examples, the system 100 can anonymize, redact, randomize, or otherwise transform portions of the interaction data so that the generated assessment object tests a concept from the interaction without exposing unnecessary identifying details. This can improve security and/or privacy.

The system 100 can use feedback loop 190 to provide validator feedback to the generation model 120. If the validator model 130, the mathematical verification model 135, or another validator returns a REJECTED status (e.g., indicating a validation score or quality score or accuracy score below a validation threshold or accuracy threshold or quality threshold), the system 100 can provide the rejection status, a reason, requested changes, unresolved-reference data, calculation data, quality-metric data, and/or recommended changes to the generation model 120 as context for regeneration or refinement of at least a portion of the structured object 125. If the validator model 130, the mathematical verification model 135, or another validator returns an APPROVED status (e.g., indicating a validation score or quality score or accuracy score above a validation threshold or accuracy threshold or quality threshold), and a consensus approval condition is satisfied, the system 100 can proceed to downstream operations. A consensus approval condition can require approval by all validator models, approval by a threshold number of validator models, approval by specified validator types, successful content verification, successful citation verification, successful deterministic verification, satisfaction of quality metrics, or combinations thereof.

The reference tracker 150 can store a forensic audit trail, including citation identifiers, annotation identifiers, model identities, verification statuses, timestamps, feedback records, generation-session identifiers, prompt hashes, response hashes, reference-source constraints, and/or mappings between cited material and portions of the structured object 125. The reference tracker 150 can verify references to reduce unsupported or hallucinated citation data, and can store whether a cited source satisfies recency, geography, peer-review, open-access, guideline-source, paywall, or other source-quality criteria. The explanation engine 155 can generate abbreviated explanations, full explanations, correct-answer explanations, incorrect-answer explanations, teaching-point explanations, and/or multi-tiered explanations. The reference tracker 150 can store references initially proposed by the generation model 120, references found by validator models 130, and references selected as replacements for less accurate, less current, or less authoritative references. A validator model 130 can determine whether a cited journal, guideline, web page, or source record is accurate, peer-reviewed, current, legitimate, published, draft-only, or otherwise suitable for the generated assessment object. The reference tracker 150 can also be referred to as a citation tracker, evidence tracker, provenance tracker, source ledger, reference ledger, citation-verification store, or evidence-mapping service. The stored mapping between cited material and portions of the structured object 125 can improve traceability by allowing a validator or later revalidation process to identify exactly which sentence, field, answer option, or explanation was supported by a particular source.

The symbol assignment and watermarking engine 140 can assign user-facing response labels after approval and can store a mapping between a user identifier, an assessment identifier, and a correct response label. In some examples, label assignment and answer ordering occur during quizzing, session assembly, presentation, or distribution rather than during generation. By deferring label assignment until after validation, the system 100 can support randomized answer presentation, label-agnostic generation, avoidance of model bias toward a particular answer letter, and forensic tracing of distributed assessment objects. The stored mapping can also include a question order, response-option order, answer-label order, or selected decoy item pattern that forms a watermark signature for a quiz, examination, or distributed study set. The watermark signature can be compared against a later copy of the quiz or examination to determine whether the later copy corresponds to the same distributed version. The symbol assignment and watermarking engine 140 can also be referred to as a label-assignment engine, answer-ordering engine, presentation-mapping engine, forensic-signature engine, distribution-tracking engine, or assessment-versioning engine.

The system 100 can include a model registry and provider-configuration store that maps pipeline roles to model identifiers, providers, model capabilities, credentials or credential references, rate-limit data, context-window limits, and enabled tool interfaces. The pipeline roles can include assessment-content generation, response-option generation, validation, explanation generation, reference verification, image generation, audio generation, video generation, augmented reality scene generation, study book generation, local verification, reranking, and scoring. The model registry can allow a model assigned to a role to be changed at runtime through an administrator interface without changing pipeline logic, and the declared capabilities can cause the system 100 to route web-search tasks, code-execution tasks, web-fetch tasks, local-model tasks, and media-generation tasks to compatible models or tools.

The reference tracker 150 can store annotation objects using deduplicated identifiers, such as identifiers based on a counter, a content hash, a citation hash, or combinations thereof. Before storing a new annotation object, the reference tracker 150 can compare the candidate annotation object with stored annotation objects and return an existing identifier when identical or equivalent content is already stored. The structured object 125 can store annotation identifiers rather than repeated full annotation content, and can maintain separate per-model annotation fields for generation-stage references, validation-stage references, explanation-stage references, and local-verification references.

In some examples, the reference tracker 150 performs sentence-aware reference verification. The reference tracker 150, mathematical verification model 135, validator model 130, or another verification service can fetch content from a reference identified by a uniform resource locator (URL), split the content into sections at sentence boundaries, provide one section in full to a local verification model while providing compressed representations of other sections, and determine whether the fetched content supports a portion of the structured object 125. Performing the verification using a local model executing on local hardware can reduce external transmission of reference content, reduce network latency, and allow large reference documents to be processed within accelerator memory limits.

The reference tracker 150 can also track formula-verification objects separately from citation-verification objects. A formula-verification object can identify a formula, input values, units, constants, a calculation precision, a result, a rounded result, a verification status, and a model or tool identity. Formula-verification objects can be stored for eGFR, creatinine clearance, body mass index, body surface area, anion gap, corrected calcium, medication dose adjustments, age-based dosing, kidney-function dosing, liver-function dosing, genotype-based dosing, and other values derived from one or more input variables.

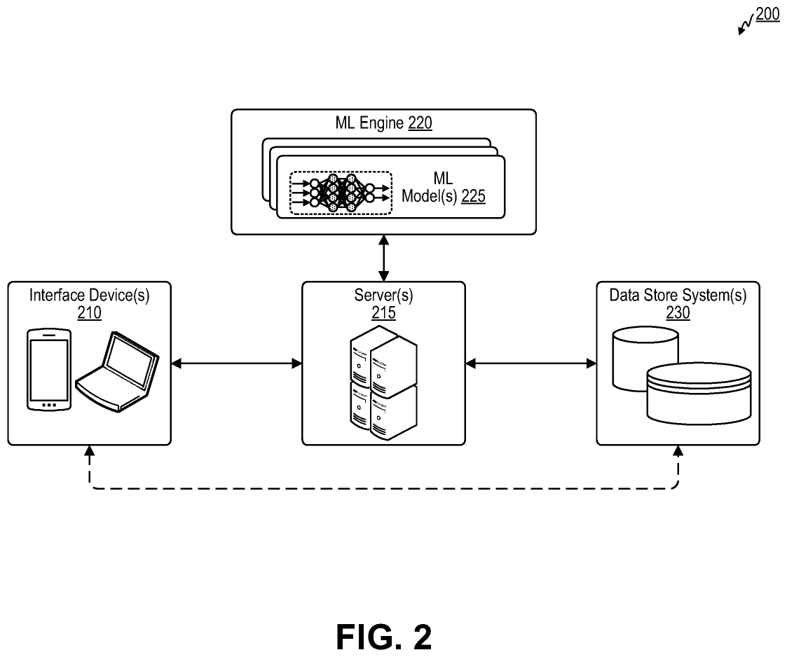

FIG. 2 is a block diagram illustrating an architecture of a system 200 for machine-learning-model-based assessment generation and interfacing. The system 200 includes interface device(s) 210, server(s) 215, an ML engine 220 that manages (e.g., trains, uses, updates) ML model(s) 225, and data store system(s) 230.

In FIG. 2, system 200 illustrates an example deployment architecture for implementing one or more portions of the system 100. The interface device(s) 210 can include mobile devices, desktop computers, tablets, workstations, augmented-reality devices, virtual-reality devices, wearable devices, clinical workstations, administrator terminals, API clients, or other devices through which prompts, parameters, media content, responses, feedback, or assessment outputs are received or presented.

The server(s) 215 can execute one or more services of the assessment generation and validation system, including prompt enrichment services, model orchestration services, validation services, deterministic verification services, reference-tracking services, scoring services, label-assignment services, watermarking services, and/or API services. The ML engine 220 can manage, select, execute, train, fine-tune, update, or otherwise coordinate the ML model(s) 225. The ML model(s) 225 can include a generation model, validator model, mathematical verification model, explanation model, embedding model, reranking model, classifier, local model, remote model, or provider-hosted model. The data store system(s) 230 can store prompts, enriched prompts, structured assessment objects, performance data, clinical parameters, question parameters, pedagogical context, media content, references, annotations, verification records, model outputs, feedback records, scores, mappings generated by the symbol assignment and watermarking engine, model configuration records, and audit logs. The data store system(s) 230 can also store previously generated assessment objects, rejected assessment objects, approved assessment objects, negative examples, and versioned assessment-object histories for later retrieval, revalidation, training, or comparison. A previously generated assessment object can be retrieved ad hoc (e.g., from the data store system(s) 230) when it is relevant to a teacher request, learner request, clinician request, current topic, current patient context, or current assessment session. The data store system(s) 230 can store an object graph or relational mapping that links prompts, enriched prompts, structured objects, validator outputs, references, user-interface events, and scores for a given generation session. Such linked storage can improve auditability and reduce duplicate processing by allowing later operations to retrieve a previously validated field or reference record instead of regenerating the same data.

In some examples, the interface device(s) 210 communicate with the server(s) 215 over one or more networks. The server(s) 215 can retrieve data from the data store system(s) 230, invoke the ML engine 220 and ML model(s) 225, and return an assessment object or interactive assessment interface to the interface device(s) 210. In other examples, at least a portion of the ML engine 220 or ML model(s) 225 is executed locally on interface device(s) 210, such as for edge-based validation, offline assessment generation, or low-latency scoring.

The server(s) 215 can include physical servers, virtual machines, containers, serverless functions, graphics-processing-unit (GPU) servers, tensor-processing-unit (TPU) servers, neural-processing-unit (NPU) devices, field-programmable gate array (FPGA) devices, database servers, cache servers, queue servers, and object-storage servers. The data store system(s) 230 can include a relational database, document database, vector database, key-value store, file store, object store, audit-log store, model registry, prompt-cache store, response-cache store, annotation store, and generation-status store. A message queue, event bus, or workflow engine can coordinate long-running generation sessions across multiple workers while preserving generation-session identifiers and cancellation state. The queue servers, event buses, or workflow engines can assign different portions of a batch to different workers while maintaining per-object lock records and generation-session identifiers. This can permit parallel processing of unresolved assessment objects while avoiding writes to assessment objects that have already been approved or locked.

The interface device(s) 210 can include sensor interfaces and media interfaces. For example, interface device(s) 210 can receive eye-tracking data, gaze data, pupil-size data, camera data, microphone data, audio data, video data, touch data, haptic-input data, controller data, keyboard data, mouse data, stylus data, physiological sensor data, heart-rate data, blood-pressure data, oxygen-saturation data, skin-moisture data, electroencephalography (EEG) data, other neurophysiological data, and/or stress, mood, attention, or confidence inputs entered through a user interface. The system 200 can use such data, when available and authorized, to personalize assessment selection, remediation, pacing, media modality, and feedback timing.

Communications among the interface device(s) 210, server(s) 215, ML engine 220, ML model(s) 225, and data store system(s) 230 can use encrypted network channels, authenticated API calls, role-based access controls, tenant isolation, sandboxed tool execution, and audit logging. In some examples, sensitive student-performance records, medical-context records, annotations, embeddings, prompt hashes, response hashes, and model-output records are stored with encryption at rest and transmitted with encryption in transit. In some examples, field-level encryption, tenant-specific keys, access-control lists, or redaction policies can be applied differently to clinical context, student-performance data, reference annotations, and model prompts. Separating these security controls by data type can reduce exposure of sensitive fields during retrieval, validation, and model-provider routing.

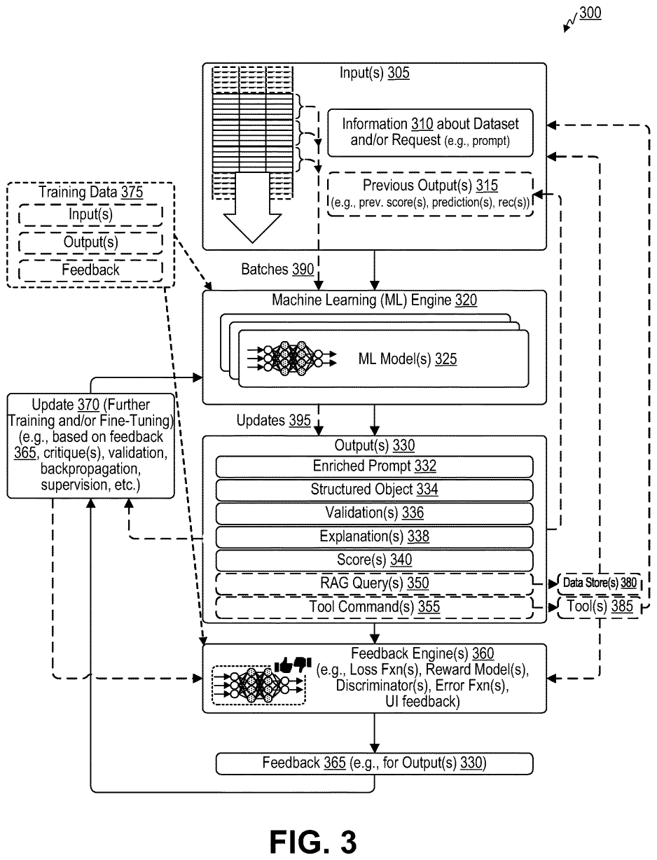

FIG. 3 is a block diagram illustrating an example of an artificial intelligence (AI) system (AI system 300) for training, use of, and/or updating of one or more machine learning (ML) model(s) (ML model(s) 325) that are used to generate output(s) 330 including an enriched prompt 332, a structured object 334, validation(s) 336, explanation(s) 338, score(s) 340, retrieval-augmented generation (RAG) query(s) 350, and/or tool command(s) 355. The AI system 300 includes an ML engine 320 that generates, trains, validates, uses (e.g., via inference), and/or updates one or more ML model(s) 325.

As shown in FIG. 3, the AI system 300 can include input(s) 305 provided to the ML engine 320 and/or the ML model(s) 325. The input(s) 305 can include information 310 about a dataset and/or request, such as a prompt, original prompt, clinical parameter set, student-performance record, question parameter set, pedagogical context record, media content item, assessment request, data from interface device(s) 210, sensor data, learner-preference data, teacher-preference data, or API request. The input(s) 305 can also include previous output(s) 315, such as a previously generated enriched prompt, structured object, validation, explanation, score, status, tool result, citation-verification result, reference-location request, model-to-model communication record, or feedback item. By using previous output(s) 315 as input, the AI system 300 can perform iterative generation, validation, regeneration, refinement, explanation generation, scoring, status generation, and selection of a next assessment object. The previous output(s) 315 can also include model-to-model messages in which a generator asks a validator for reference assistance, a validator requests clarification from a generator, or one model identifies an inconsistency in a prompt, response, reference, calculation, or explanation generated by another model.

The validation(s) 336 can include approval statuses, rejection statuses for one or more assessment objects in a batch, validator comments, validator scores, citation-verification results, mathematical-verification results, format-verification results, medical-accuracy results, consensus records, and/or reasons for rejection. In some examples, the validation(s) 336 are generated by a plurality of independent validator models that audit the structured object 334 without directly modifying the structured object 334. In some examples, the validation(s) 336 verify that none of the incorrect response-option objects could accurately be interpreted as correct given the context of the question, and/or that the correct response-option object cannot accurately be interpreted as incorrect given the context of the question.

Any assessment object, explanation object, media object, or derivative content object can reenter a validation and regeneration loop as part of a revalidation process. The revalidation process can use new references, updated source material, new models, new validators, updated reference-source constraints, updated mathematical formulas, updated user feedback, or changed performance data to determine whether a previously generated object should be approved, rejected, regenerated, deactivated, reactivated, or updated.

The data used for assessment-object generation, content-object generation, validation, or revalidation can originate from web searches, reference databases, expert interviews, books, articles, guideline documents, audio material, video material, pictorial material, radiographs, roentgen images, electromagnetic-signal recordings, temperature sensors, any authorized sensor, EEG signals, interface device(s) 210, data store system(s) 230, data store(s) 380, or another data source. Retrieved or received source material can be summarized, embedded, indexed, cited, verified, and stored with provenance metadata before being incorporated into an enriched prompt or validation record.

The AI system 300 can use feedback engine(s) 360 to generate feedback 365 for the output(s) 330. The feedback engine(s) 360 can include loss functions, reward models, discriminators, error functions, user-interface feedback processors, validation processors, or other feedback mechanisms. The update 370 can include further training, fine-tuning, prompt update, model-configuration update, threshold update, retrieval-index update, or rules update based on feedback 365, critique, validation, backpropagation, supervision, reinforcement, or another update signal. Training data 375 can be used to train or fine-tune the ML model(s) 325. Data store(s) 380 can store training data 375, prompts, structured objects, references, indexes, feedback, model outputs, annotations, and audit records. Tool(s) 385 can execute the tool command(s) 355 and return results that are used as input(s) 305, previous output(s) 315, feedback 365, or portions of the output(s) 330. Batch records 390 can include grouped requests, grouped assessment objects, grouped explanation objects, grouped media objects, grouped categories, or grouped training records, and updates 395 can include incremental updates to output(s), model parameters, audit records, scores, pass/fail statuses, approval/rejection statuses, or batch records as data is processed. The feedback 365 and training data 375 can include rejected assessment objects and rejected response-option objects as negative training data, so that one or more ML model(s) 325 learn to avoid repeating a rejected content pattern, unsupported citation pattern, answer-choice imbalance, or calculation error. In some examples, the training data 375 includes simulated patient datasets generated from feature-parameter records, outcome records, source-seed records, expert-input records, or combinations thereof, and the simulated patient datasets can be used without storing or exposing corresponding real-patient records. A simulated patient dataset can include simulated feature values, simulated outcomes, metadata, source-seed identifiers, expert identifiers, dataset reputation values, and validation statuses that are stored in the data store(s) 380 for training, fine-tuning, evaluation, or prompt enrichment. Because the simulated patient datasets can be generated according to ranges, distributions, thresholds, categorical values, or outcome-specific feature relationships, the AI system 300 can increase training coverage for rare clinical presentations while reducing access to sensitive patient information.

The ML model(s) 325 can include, for instance, one or more machine-learning architectures and/or algorithms such as neural networks (e.g., artificial neural networks (ANNs), convolutional neural networks (CNNs), time-delay neural networks (TDNNs), deep networks, feed-forward networks, recurrent neural networks (RNNs), and transformer-based models), autoencoders (AEs) and/or variational autoencoders (VAEs), deep belief networks (DBNs), generative models (e.g., generative adversarial networks (GANs) and/or conditional GANs (cGANs), diffusion models, and/or autoregressive models), sequence-to-sequence (Seq2Seq) models, and/or transformer-based language models (e.g., decoder-only, encoder-decoder, or encoder-only variants), which may be configured as mixtures-of-experts (MoE), may be instruction-tuned and/or alignment-tuned, and may be multimodal (e.g., jointly processing text with images, audio, video, and/or tabular features). In some examples, the ML model(s) 325 may include encoder models that generate embeddings for retrieval and similarity search, graph neural networks for relational data, and/or other types of ML models such as support vector machines (SVMs), random forests (RFs), gradient-boosted decision trees, and/or generalized linear models. In some examples, the ML model(s) 325 may include a U-Network (U-Net) architecture having a contracting path and an expansive path, and may include convolution, up-convolution, pooling, and skip connections to extract complex features while preserving and reconstructing spatial information. The ML model(s) 325 may be deployed as a single model, an ensemble of models, and/or a pipeline of models (e.g., an embedding model feeding a retriever and a generator), and may support supervised, unsupervised, semi-supervised, self-supervised, reinforcement, and/or online/continual learning.

In some examples, one or more of the ML model(s) 325, feedback engine(s) 360, retrieval models, ranking models, statistical models, or rule-based selectors can select an assessment object for a user based on prior assessment results, a learning path, category history, teacher preferences, learner preferences, sensor data, target examination, predicted score improvement, desired retention interval, or another personalization signal.

In some examples, any other artificial intelligence (AI) systems disclosed herein (e.g., AI algorithms, ML models, specific types of algorithms or models such as any of those listed above, or combinations thereof), can include the AI system 300, the ML engine 320, the ML model(s) 325, and/or the feedback engine(s) 360, or vice versa.

Within FIG. 3, a graphic representing the ML model(s) 325 illustrates a set of circles connected to one another and organized in an array. Each of the circles can represent a node, a neuron, a perceptron, a layer, a portion thereof, or a combination thereof. In some examples, each of the circles (e.g., the nodes, neurons, and/or perceptrons) can correspond to specific hardware that stores and/or processes data on behalf of that node, neuron, and/or perceptron. Such hardware can include data storage element(s) (e.g., registers, flip-flops, on-chip memory, non-transitory computer-readable storage media, and/or other types of memory), processing element(s) (e.g., microprocessors, application-specific integrated circuits (ASICs), field programmable gate arrays (FPGAs), processors, controllers, and/or other types of processing elements), input connector(s), output connector(s), or combinations thereof.

The circles are arranged (within the array) in columns, which represent layers. The leftmost column of white circles represents an input layer. The rightmost column of white circles represents an output layer. Two columns (layers) of shaded circles between the leftmost column of white circles and the rightmost column of white circles each represent hidden layers. An ML model can include more or fewer hidden layers than the two illustrated, but includes at least one hidden layer. In some examples, the layers and/or nodes represent interconnected filters, and information associated with the filters is shared among the different layers with each layer retaining information as the information is processed. The lines between nodes can represent node-to-node interconnections along which information is shared. The lines between nodes can also represent weights (e.g., numeric weights) between nodes, which can be tuned, updated, added, and/or removed as the ML model(s) 325 are trained and/or updated. In some examples, each of the lines between nodes can correspond to specific hardware that stores, updates, and/or applies such weights, for instance including synaptic circuits that can include data storage element(s) (e.g., registers, flip-flops, on-chip memory, non-transitory computer-readable storage media, and/or other types of memory) for storing weights, processing element(s) (e.g., microprocessors, application-specific integrated circuits (ASICs), field programmable gate arrays (FPGAs), processors, controllers, and/or other types of processing elements), input connector(s), output connector(s), or combinations thereof. In some cases, certain nodes (e.g., nodes of a hidden layer) can transform the information of each input node by applying activation functions (e.g., filters) to this information, for instance applying convolutional functions, downscaling, upscaling, data transformation, and/or any other suitable functions. In some examples, information is received, processed, and ultimately output in a flow from left to right through the layers of nodes, neurons, and/or perceptrons in the illustrated array. The weights can be referred to as synaptic weights and/or numeric weights, and can each represent the strength of a connection between two neurons.

In some examples, the ML model(s) 325 can include a feed-forward network, in which case there are no feedback connections where outputs of the network are fed back into itself. In some cases, the ML model(s) 325 can include a recurrent neural network, which can have loops that allow information to be carried across nodes while reading in input. In some cases, the network can include a convolutional neural network, which may not link every node in one layer to every other node in the next layer.

In some examples, the ML engine 320 may implement an inference runtime that improves the operation of the AI system 300 by reducing latency, memory utilization, network overhead, and/or compute cycles associated with generating the output(s) 330. For example, the inference runtime may perform request batching or micro-batching, caching of intermediate activations and/or prior inference results, scheduling of workloads across heterogeneous compute resources (e.g., central processing units (CPUs), graphics processing units (GPUs), tensor processing units (TPUs), or accelerators), and/or selection among multiple model variants based on resource constraints or service-level objectives. In further examples, the ML model(s) 325 may be optimized for deployment using quantization, pruning, distillation, compilation, or operator fusion, thereby reducing memory bandwidth and improving throughput while maintaining target accuracy. In some examples, the inference runtime enforces deterministic or constrained decoding (for generative models) and/or structured output validation, thereby reducing invalid outputs and avoiding repeated recomputation or retry loops. The inference runtime can also reuse key-value caches, prompt-prefix caches, embedding caches, and schema-validation results across related assessment objects in a batch. In some examples, constrained decoding can enforce field names, field order, enumerated status values, numeric ranges, or required reference identifiers in the structured object 334.

One or more input(s) 305 can be provided to the ML model(s) 325. The ML model(s) 325 can be trained by the ML engine 320 (e.g., based on training data 375) to generate one or more output(s) 330. In some examples, the input(s) 305 include information 310. The information 310 can include information received through an interactive user interface, such as a physiological measurement, eye tracking, EEG signals, electromagnetic sensor signals captured without contacting a skin surface, past learner performance, prompt, request, clinical parameter set, question parameter set, student-performance record, pedagogical context record, reference identifier, media item, assessment response, or another type of input that is based on at least one user interface input. In some examples, the input(s) 305 can include information retrieved from data store(s) 380, for instance via retrieval-augmented generation (RAG) using RAG query(s) 350. In some examples, the input(s) 305 can include information generated using tool(s) 385, for instance based on use of tool command(s) 355. In some examples, the input(s) 305 can include prompt(s) that are modified and/or enhanced using information retrieved from data store(s) 380, for instance via RAG query(s) 350 and/or tool command(s) 355 to tool(s) 385.

The output(s) 330 that ML model(s) 325 generate by processing the input(s) 305 (e.g., the information 310 and/or the previous output(s) 315) can include the enriched prompt 332, the structured object 334, the validation(s) 336, the explanation(s) 338, the score(s) 340, the RAG query(s) 350, and the tool command(s) 355. The enriched prompt 332 can include a prompt augmented with student-performance data, clinical parameters, question parameters, pedagogical context, media content, retrieved reference material, formatting constraints, and/or model-specific instructions. The structured object 334 can include a structured assessment object, such as a JSON object, having an assessment content portion (e.g., a question), a correct response-option object (e.g., a correct response to the question), one or more incorrect response-option objects (e.g., response options to the question that are incorrect but might appear plausible), citations, metadata, and/or explanation fields. In some examples, the ML model(s) 325 can generate the incorrect response-option objects to be correct response-option objects from other questions. That is, the ML model(s) 325 can generate incorrect response options for a first question by choosing response options that are correct for a second (different) question, but that are incorrect for the first question. This can ensure that the incorrect response options sound plausible. The structured object 334 can also be described at different levels of breadth as a structured content object, assessment record, item schema, generated payload, validation-ready payload, question-response record, or machine-readable assessment record.

In some examples, the ML engine 320 is configured to retrieve an existing assessment object from the data store(s) 380, generate a new assessment object when no suitable stored object is available, or generate a new assessment object when a freshness, personalization, preference, or revalidation rule favors newly generated content over stored content. For example, when a learner or teacher requests cardiology material, the ML engine 320 can retrieve a first subset of previously generated cardiology assessment objects from the data store(s) 380 and generate a second subset of new cardiology assessment objects for the same session. Combining retrieved and newly generated objects can reduce model calls while preserving freshness, because stored objects can be reused when current and new objects can be generated only for missing topics, stale references, or changed learner needs.

The validation(s) 336 can include approval statuses, rejection statuses for one or more assessment objects in a batch, validator comments, validator scores, citation-verification results, mathematical-verification results, format-verification results, medical-accuracy results, consensus records, and/or reasons for rejection. In some examples, the validation(s) 336 are generated by a plurality of independent validator models that audit the structured object 334 without directly modifying the structured object 334. In some examples, the validation(s) 336 verify that none of the incorrect response-option objects could accurately be interpreted as correct given the context of the question (e.g., to avoid an answer where two or more response options could be valid correct answers), and/or that the correct response-option object cannot accurately be interpreted as incorrect given the context of the question. A consensus record can indicate that all validator models approved the structured object 334, that a threshold number of validator models approved the structured object 334, or that a threshold score was satisfied even if fewer than all validator models returned approval statuses. For instance, the validation(s) 336 can indicate approval when two out of three validator models approve, when an average validation score exceeds a threshold, when a minimum validator score exceeds a threshold, or when a maximum validator score from a designated validator role exceeds a threshold. The consensus record can also be referred to as a quorum record, approval ledger, validator-vote record, score-aggregation record, validation ledger, or quality-control state.

In some examples, validator models can generate field-specific validator feedback for any portion of the structured object 334, associated references, annotations, explanations, or metadata. Depending on role permissions, the feedback can include suggested replacement text, citation updates, annotation updates, or regeneration instructions. In comment-only examples, such changes are applied by a generation model, refinement model, or controlled update service after a validation status is recorded.

The explanation(s) 338 can include abbreviated explanations, full explanations, teaching-point explanations, correct-answer explanations, incorrect-answer explanations, and/or remediation explanations. The score(s) 340 can include an assessment score, item score, confidence score, validation score, quality score, difficulty score, similarity score, learner-performance score, answer-correctness score, or other computed metric. In some examples, the score(s) 340 can refer to score(s) or grade(s) of a test, exam, quiz, or other assessment that includes questions and/or response options generated as part of the structured object 334—for instance indicating how many questions a user got correct (right) or incorrect (wrong). The ML model(s) 325 can generate each of the output(s) 330 based on the information 310, information from the data store(s) 380, and/or other types of input(s) 305 (e.g., previous output(s) 315). In some examples, the score(s) 340 can refer to validation or verification score(s) (e.g., quality scores, accuracy scores) generated by validator models, such as the validator model 130 and/or the mathematical verification model 135.

The RAG query(s) 350 can include search queries, vector-search queries, keyword queries, database queries, filter criteria, metadata constraints, citation requests, or other retrieval instructions for retrieving source material, clinical guidelines, prior assessment objects, student-performance records, media items, or other context from the data store(s) 380 and/or external reference sources. The tool command(s) 355 can include structured instruction(s), function call(s), procedure call(s), message(s), request(s), and/or other command data generated by the ML model(s) 325 and/or the ML engine 320 to cause one or more tool(s) 385 to perform action(s).

The tool(s) 385 can include software or hardware tool(s), service(s), application(s), agent(s), workflow engine(s), data store(s), reference database(s), code-execution tool(s), calculator(s), citation-verification tool(s), document-processing tool(s), media-generation tool(s), rendering tool(s), scoring tool(s), learning-management-system interface(s), and/or annotation system(s) that can be invoked using the tool command(s) 355. The ML engine 320 and/or ML model(s) 325 can interface with the tool(s) 385 using one or more tool interface technologies, such as application programming interface(s) (APIs), webhook(s), message queue(s), event bus(es), plugin interface(s), browser automation interface(s), database interface(s), command-line interface(s) (CLIs), Model Context Protocol (MCP) interface(s) and/or server(s), Agent Communication Protocol (ACP) interface(s), agent-to-agent protocol(s), or a combination thereof. In some examples, an adapter, gateway, sandbox, permission boundary, policy engine, guardrail, validator, orchestrator, or other interface layer routes and/or validates the tool command(s) 355 before the tool command(s) 355 reach the tool(s) 385. Result(s) returned by the tool(s) 385 can be routed back to the ML engine 320 and/or ML model(s) 325 as input(s) 305, previous output(s) 315, feedback 365, validation(s) 336, RAG query results, or tool-status records.

In some examples, the ML model(s) 325 generate the tool command(s) 355 proactively and/or automatically when the validation(s) 336 indicate that additional verification is needed, when the score(s) 340 satisfy threshold(s), when a confidence value satisfies a threshold, when a RAG query 350 should be executed, and/or when the structured object 334 requires rendering, citation tracking, label assignment, watermarking, or storage. For example, a tool command 355 can invoke a calculator to verify a numerical value, query a reference database, store an audit record, render an assessment interface, update a student-performance record, or request an additional validation pass. The tool command(s) 355 can be executed automatically after a trigger is satisfied or queued for review, approval, simulation, dry-run execution, or delayed execution.

In some examples, result(s) returned by the tool(s) 385 can be used as additional input(s) 305, previous output(s) 315, and/or feedback 365. For instance, a tool response can indicate whether a tool command 355 was syntactically or semantically valid, authorized, executable, successful, rejected, rolled back, duplicative, ill-timed, unsafe, inconsistent with a current tool state, and/or otherwise appropriate. Such information can indicate whether the tool command 355, validation(s) 336, explanation(s) 338, score(s) 340, and/or RAG query(s) 350 were accurate, reliable, calibrated, useful, and/or aligned with an intended purpose.

In some examples, the ML model(s) 325 and/or a pre-processor can perform data formatting, conversion, normalization, and/or standardization operations. For instance, the input(s) 305 can be received from multiple data structures, multiple types of data structures, multiple devices, and/or multiple formats, including databases, tables, linked lists, arrays, dictionaries, spreadsheets, text extracted from documents, slide decks, electronic medical-record exports, question-bank records, student-performance logs, media files, recordings of patient-provider encounters, synthetic records, simulated patient charts, simulated medical histories, and simulated assessment histories. To improve accuracy of the output(s) 330, the ML model(s) 325 and/or a pre-processor can convert data from a first format to a second format, normalize formats, convert non-standardized data entries into a standardized format, translate text from a first language to a second language, normalize units or laboratory-value formats, or a combination thereof. This pre-processing can allow data from different sources, formats, and/or devices to interoperate and be integrated to generate the enriched prompt 332, structured object 334, validation(s) 336, explanation(s) 338, score(s) 340, RAG query(s) 350, and/or tool command(s) 355. In some examples, the ML model(s) 325 and/or a pre-processor can improve security and/or privacy by tokenizing, redacting, encrypting, obfuscating, and/or transforming sensitive fields prior to inference; encrypting feature vectors, embeddings, and/or retrieved content at rest and/or in transit; enforcing tenant-and role-based access controls for retrieval from the data store(s) 380; and/or applying policy-based filtering so that retrieval-augmented content is constrained to authorized sources. Formatting and conversion operations can also convert speech to text, text to speech, a quiz to a flashcard set, a multiple-choice object to a true-or-false object, a document to a slide deck, or an audio or video record to a text record used for prompt enrichment or validation. For example, laboratory values can be normalized into a common unit system, drug names can be mapped to normalized identifiers, and audio from an encounter can be converted into timestamped text before the enriched prompt 332 is generated.

In some examples, the ML model(s) 325 can identify something in the input(s) 305 about which the data store(s) 380 include additional information, and can fashion at least one query (e.g., the RAG query(s) 350) for the data store(s) 380 to retrieve the additional information from the data store(s) 380. For instance, if the information 310 references a clinical condition, laboratory value, question category, learning objective, prior missed tag, citation, or media item, the RAG query(s) 350 can include one or more queries of the data store(s) 380 for additional information about that condition, value, category, objective, tag, citation, or media item. The additional information retrieved from the data store(s) 380 using the RAG query(s) 350 can be used as part of the input(s) 305 (e.g., as part of the information 310 and/or part of the previous output(s) 315) for further passes of data processing by the ML model(s) 325.

In examples that employ RAG, the AI system 300 may improve computational efficiency and output reliability by maintaining one or more searchable indexes over the data store(s) 380, such as a vector index for approximate nearest-neighbor (ANN) retrieval, a lexical/inverted index, or a hybrid index combining both. The ML engine 320 may generate embeddings for the query(s) 350 and/or for candidate documents, retrieve a top-K set of candidates using the index, and optionally apply a re-ranking stage (e.g., a cross-encoder or scoring model) before incorporating selected passages into model inputs. In some examples, the AI system 300 caches retrieved results (e.g., keyed by an embedding hash, tenant identifier, and/or policy context) to reduce repeated index lookups and network round trips, and attaches provenance metadata (e.g., document identifiers, timestamps, and/or citation pointers) to retrieved content used to produce the output(s) 330, thereby improving auditability and traceability. The searchable indexes can be partitioned or filtered by geography, specialty, publication date, peer-review status, institution, tenant, course, learner cohort, or reference-source criterion before retrieval is performed. Filtering before retrieval can reduce irrelevant candidate passages and can reduce the number of tokens provided to a generation or validation model.

In some examples, certain output(s) 330 (e.g., the enriched prompt 332, structured object 334, validation(s) 336, explanation(s) 338, score(s) 340, RAG query(s) 350, and/or tool command(s) 355) can be used as part of the input(s) 305 to the ML model(s) 325 (e.g., as previous output(s) 315) for identifying other output(s) 330. For instance, the structured object 334 can be processed as previous output(s) 315 to generate validation(s) 336, the validation(s) 336 can be processed as previous output(s) 315 to generate RAG query(s) 350 or tool command(s) 355, and the score(s) 340 can be processed as previous output(s) 315 to generate explanation(s) 338 or select a subsequent assessment object. In some examples, dependencies among output(s) 330 are stored as a directed graph, so that the AI system 300 can identify which downstream validations, explanations, scores, or tool commands should be regenerated when one upstream field changes.

In some examples, the ML model(s) 325 can generate intermediate data based on the input(s) 305, and can generate the output(s) 330 based on the intermediate data. For instance, the ML model(s) 325 can process the information 310 to identify a learner profile, a target difficulty, a clinical concept, a question category, candidate citations, missing validation information, similar prior assessment objects, or media-context information, and can then generate the output(s) 330 based on this intermediate data to improve personalization, traceability, accuracy, and/or difficulty calibration.

In some examples, the ML system repeats the process illustrated in FIG. 3 multiple times to generate the output(s) 330 in multiple passes, using some of the output(s) 330 from earlier passes as some of the input(s) 305 in later passes (e.g., as some of the previous output(s) 315). For instance, in a first illustrative example, in a first pass, the ML model(s) 325 can generate the enriched prompt 332 based on input of the information 310 into the ML model(s) 325. In a second pass, the ML model(s) 325 can generate the structured object 334 based on the enriched prompt 332. In a third pass, the ML model(s) 325 can generate validation(s) 336, explanation(s) 338, score(s) 340, RAG query(s) 350, and/or tool command(s) 355 based on the structured object 334 and other previous output(s) 315. The multiple passes can be implemented as pipeline stages, workflow states, queue jobs, agent tasks, model-role calls, or service invocations that exchange structured intermediate data.

In another illustrative example, a later pass can identify tool command(s) 355 based on the information 310, validation(s) 336, explanation(s) 338, score(s) 340, RAG result(s), and/or tool result(s). For example, when validation(s) 336 indicate an unverified citation, the ML model(s) 325 can generate a tool command 355 that causes a tool 385 to retrieve or verify the citation. When a score 340 reaches or crosses a threshold, the ML model(s) 325 can generate a tool command 355 that causes a tool 385 to update a student-performance record, select a next assessment object, or present a remediation explanation. In some examples, the mathematical verification model 135 can generate tool command(s) 355 to tool(s) 385 that are used to perform mathematical calculations, since language models are sometimes inaccurate in calculations, but can be improved to be more accurate through access to calculators, custom code, and/or other tool(s) 385 designed for the task. The tool command(s) 355 can include idempotency keys, timeout values, retry limits, rollback instructions, and permission scopes so that failed or duplicated tool calls do not create inconsistent assessment records.

In some examples, the ML system includes one or more feedback engine(s) 360 that generate and/or provide feedback 365 about the output(s) 330. In some examples, the feedback 365 indicates how well the output(s) 330 align to corresponding expected output(s), how well the output(s) 330 serve their intended purpose, or a combination thereof. In some examples, the feedback engine(s) 360 include loss function(s), reward model(s) (e.g., other ML model(s) that are used to score the output(s) 330), discriminator(s), error function(s) (e.g., in back-propagation), user interface feedback received via a user interface from a user, or a combination thereof. In some examples, the feedback 365 can include one or more alignment score(s) that score a level of alignment between the output(s) 330 and the expected output(s) and/or intended purpose.

In some examples, the feedback 365 itself can be generated by another AI model, ML model, large language model, multimodal model, natural-language-processing model, non-natural-language-processing model, statistical model, rule-based system, or combination thereof.

The ML engine 320 of the ML system can update (e.g., further train and/or fine-tune) the ML model(s) 325 based on the feedback 365 to perform an update 370 (e.g., further training and/or fine-tuning) of the ML model(s) 325 based on the feedback 365. In some examples, the feedback 365 includes positive feedback, for instance indicating that the output(s) 330 closely align with expected output(s) and/or that the output(s) 330 serve their intended purpose. In some examples, the feedback 365 includes negative feedback, for instance indicating a mismatch between the output(s) 330 and the expected output(s), and/or that the output(s) 330 do not serve their intended purpose. For instance, high amounts of loss and/or error (e.g., exceeding a threshold) can be interpreted as negative feedback, while low amounts of loss and/or error (e.g., less than a threshold) can be interpreted as positive feedback. Similarly, high amounts of alignment (e.g., exceeding a threshold) can be interpreted as positive feedback, while low amounts of alignment (e.g., less than a threshold) can be interpreted as negative feedback. In some examples, update 370 includes regenerating or reweighting training data 375 based on feedback 365, such as by increasing a sampling weight for simulated patient datasets associated with high-quality feedback and decreasing a sampling weight for simulated patient datasets associated with negative feedback. The update 370 can update model weights, prompt templates, validator thresholds, reference filters, data-selection weights, dropout configurations, parameter values, hyperparameter values, model-routing rules, or ensemble weights. The ML engine 320 can test candidate updates in an exploration phase and then deploy a selected update in an optimization phase after one or more processing characteristics, such as accuracy, latency, memory use, power use, heat generation, false-positive rate, false-negative rate, calibration error, or validation-pass rate, satisfy a threshold.

In response to positive feedback, the ML engine 320 can perform update 370 to strengthen and/or reinforce weights, connections, and/or hyperparameters associated with generation of the output(s) 330, encouraging similar output(s) for similar input(s). In response to negative feedback, the ML engine 320 can weaken and/or remove such weights, connections, and/or hyperparameters to discourage similar output(s) for similar input(s). In this way, update 370 can improve accuracy of the ML model(s) 325, for instance by reducing false positive(s) and/or false negative(s).

Individual responses of learners or other users for individual assessment objects can be analyzed statistically, by one or more AI/ML models, by one or more rule-based systems, or by combinations thereof. The analysis can generate feedback 365 for an assessment object, response-option set, explanation set, category, difficulty level, or learning path.

For example, if a threshold number or threshold percentage of users answer an assessment object incorrectly, the AI system 300 can determine that the assessment object may lack clarity, specificity, adequate differentiation among response-option objects, appropriate difficulty calibration, or sufficient support in the associated explanation. In another example, if user selections are distributed nearly evenly between two or more response-option objects, the AI system 300 can determine that those response-option objects may be insufficiently differentiated. The assessment object can then be redirected for revalidation, refinement, regeneration, operator review, or deactivation.

For instance, if the score(s) 340, validation(s) 336, explanation(s) 338, RAG query(s) 350, and/or tool command(s) 355 are used in a subsequent process, such as presenting an assessment object, regenerating a structured object, validating a citation, invoking a verification tool, or updating a student-performance record, success of that process can be interpreted as positive feedback 365, while failure or lack of success can be interpreted as negative feedback 365. Either way, update 370 can improve the AI system 300 and the overall system by improving the consistency with which the subsequent process succeeds.

In some examples, the tool(s) 385 are a source of feedback 365. For instance, the feedback engine(s) 360 can receive or generate feedback 365 based on validity, policy, or permission check(s), execution result(s), exception(s), latency, retry count(s), rollback(s), downstream state change(s), audit log(s), confirmation(s), rejection(s), or other information associated with a tool command 355 and/or tool 385. Valid, authorized, successful, and/or well-timed tool command(s) 355 can provide positive feedback; invalid, unauthorized, unsuccessful, unsafe, ill-timed, rejected, or state-inconsistent tool command(s) 355 can provide negative feedback for updating, recalibrating, reweighting, or avoiding similar future output(s).

Assessment objects that are later determined to be inadequate, erroneous, poorly differentiated, outdated, unsupported by references, or otherwise below a quality threshold can remain in a data store as rejected-output records, negative examples, learner-reference records, or training records. Such records can be used for debugging, quality analysis, future training, negative training, adversarial training, or generative-adversarial-network training for models that generate or validate content. For example, a rejected cardiology question with two arguably correct answer options can be stored as a negative example for later generation prompts, validator calibration, or adversarial testing.

In some examples, the AI system 300 monitors model performance and data drift to maintain reliable operation over time. For example, the ML engine 320 and/or feedback engine(s) 360 may compute drift metrics over input feature distributions, embedding distributions, and/or output distributions, and may monitor performance metrics such as false positives, false negatives, calibration error, latency, and throughput. When drift or degradation exceeds one or more thresholds, the update 370 may trigger retraining, fine-tuning, re-calibration, selection of a different model checkpoint, and/or rollback to a prior checkpoint. In some examples, the update process is performed in a staged deployment, such as shadow mode, canary release, or A/B testing, to validate updates prior to wider rollout. In some examples, the ML engine 320 trains an auxiliary model that predicts how a candidate parameter configuration, dropout configuration, retrieval policy, prompt policy, validator quorum rule, or model-routing rule is expected to change one or more processing characteristics before the candidate configuration is applied to production assessment generation. The AI system 300 can skip, delay, or sandbox candidate updates predicted to degrade a processing characteristic, thereby reducing unnecessary training runs, inference calls, or validator calls.

In some examples, the AI system 300 can use on-premises models, local models, cloud-hosted models, provider-hosted models, external models, or combinations thereof as generating models, validating models, explanation models, ranking models, or feedback models. The ML engine 320 can train, fine-tune, tune, distill, or otherwise update such models using generated content, cross-validated approved content, rejected content, user feedback, validator feedback, or other training records.

In some examples, the ML engine 320 can also perform an initial training of the ML model(s) 325 before the ML model(s) 325 are used to generate the output(s) 330 based on the input(s) 305. During the initial training, the ML engine 320 can train the ML model(s) 325 based on training data 375. In some examples, the training data 375 includes examples of input(s) (of any input types discussed with respect to the input(s) 305), output(s) (of any output types discussed with respect to the output(s) 330), and/or feedback (of any feedback types discussed with respect to the feedback 365). In some cases, positive feedback in the training data 375 can be used to perform positive training, to encourage the ML model(s) 325 to generate output(s) similar to the output(s) in the training data given input of the corresponding input(s) in the training data. In some cases, negative feedback in the training data 375 can be used to perform negative training, to discourage the ML model(s) 325 from generating output(s) similar to the output(s) in the training data given input of the corresponding input(s) in the training data. In some examples, the training of the ML model(s) 325 (e.g., the initial training with the training data 375, update(s) 370 based on the feedback 365, and/or other modification(s)) can include fine-tuning of the ML model(s) 325, retraining of the ML model(s) 325, or a combination thereof.

In some examples, the training data 375 includes expert-provided source seeds that define possible feature values, feature distributions, outcome labels, likelihoods, recommended tests, recommended treatments, and feature-importance values for simulated clinical records. The simulated clinical records can be generated as tables, database rows, structured objects, or tensors and can include numerical values, Boolean values, categorical values, strings, Likert-scale values, missing-value indicators, and metadata. For example, the training data 375 can include simulated records for a rare condition using feature distributions for age, body mass index, laboratory values, imaging findings, symptoms, and outcomes, so that the ML model(s) 325 can be trained or evaluated even when real-world records for that condition are sparse or unavailable. In some examples, the feedback 365 modifies reputation scores or weights associated with an expert source, simulated-patient population, simulated clinical record, validator, or querying user, and the update 370 regenerates or resamples the training data 375 according to the modified reputation scores or weights. The training data 375 can therefore include privacy-preserving synthetic, simulated, anonymized, redacted, randomized, transformed, or semi-random patient-context records that support generation of assessment objects without requiring exposure of original patient identifiers.

In some examples, the ML model(s) 325 can include an ensemble of multiple ML models, and the ML engine 320 can curate and manage the ML model(s) 325 in the ensemble. The ensemble can include ML model(s) 325 that are different from one another to produce different respective outputs, which the ML engine 320 can average (e.g., mean, median, and/or mode) to identify the output(s) 330. In some examples, the ML engine 320 can calculate the standard deviation of the respective outputs of the different ML model(s) 325 in the ensemble to identify a level of confidence in the output(s) 330. In some examples, the standard deviation can have an inverse relationship with confidence. For instance, if the respective outputs of the different ML model(s) 325 are very different from one another (and thus have a high standard deviation above a threshold), the confidence that the output(s) 330 are accurate may be low (e.g., below a threshold). On the other hand, if the respective outputs of the different ML model(s) 325 are equal or very similar to one another (and thus have a low standard deviation below a threshold), the confidence that the output(s) 330 are accurate may be high (e.g., above a threshold). The ensemble can be a validator ensemble, generator ensemble, translation ensemble, reference-verification ensemble, or mixed-role ensemble that combines models having different strengths. In some examples, ensemble weights are updated based on observed performance for particular output types, such as question generation, citation verification, simulated-patient generation, medical-outcome prediction, explanation generation, translation, or sensor-based adaptation.

In some examples, the ML engine 320 may compute one or more uncertainty measures associated with the output(s) 330, such as predictive variance across an ensemble, entropy of a probability distribution, disagreement among models, or other confidence indicators. The AI system 300 may further calibrate confidence values (e.g., via temperature scaling, isotonic regression, or other calibration techniques) to better align reported confidence with observed correctness. Based on the uncertainty and/or calibrated confidence, the ML engine 320 may abstain from producing a definitive output, may route the input(s) 305 to an alternate model or a rules-based fallback, may invoke additional retrieval passes, and/or may escalate to a human review workflow. These controls can reduce erroneous outputs, reduce downstream error propagation, and improve the overall reliability of the computing system under distribution shift or adversarial/noisy inputs. Uncertainty gating can improve downstream reliability by preventing low-confidence generated content from being presented to a learner before additional verification, retrieval, or regeneration has occurred.

In some examples, different ML model(s) 325 in the ensemble can include different types of models. For instance, in some examples, an ensemble can include a neural network and a support vector machine that are both trained to process the input(s) 305 to generate at least a subset of the output(s) 330. In some examples, the ensemble may include different ML model(s) 325 that are trained to process different inputs of the input(s) 305 and/or to generate different outputs of the output(s) 330. For instance, a first model or set of models can process the input(s) 305 to generate the enriched prompt 332, a second model or set of models can process the enriched prompt 332 to generate the structured object 334, a third model or set of models can process the structured object 334 to generate validation(s) 336, and a fourth model or set of models can generate explanation(s) 338, score(s) 340, RAG query(s) 350, and/or tool command(s) 355. In some examples, the ML engine 320 can choose specific ML model(s) 325 to be included in the ensemble because the chosen ML model(s) 325 are effective at accurately processing particular types of input(s) 305, are effective at accurately generating particular types of output(s) 330, are generally accurate, process input(s) 305 quickly, generate output(s) 330 quickly, are computationally efficient, have higher or lower degrees of uncertainty than other models in the ensemble, or a combination thereof.

In some examples, one or more of the ML model(s) 325 can be initialized with weights, connections, and/or hyperparameters that are selected randomly. This can be referred to as random initialization. These weights, connections, and/or hyperparameters are modified over time through training (e.g., initial training with the training data 375 and/or update(s) 370 based on the feedback 365), but the random initialization can still influence the way the ML model(s) 325 process data, and thus can still cause different ML model(s) 325 (with different random initializations) to produce different output(s) 330. Thus, in some examples, different ML model(s) 325 in an ensemble can have different random initializations.

As an ML model (of the ML model(s) 325) is trained (e.g., along the initial training with the training data 375, update(s) 370 based on the feedback 365, and/or other modification(s)), different versions of the ML model at different stages of training can be referred to as checkpoints. In some examples, after each new update to a model (e.g., update 370) generates a new checkpoint for the model, the ML engine 320 tests the new checkpoint (e.g., against testing data and/or validation data where the correct output(s) are known) to identify whether the new checkpoint improves over older checkpoints or not, and/or if the new checkpoint introduces new errors (e.g., false positive(s) and/or false negative(s)). This testing can be referred to as checkpoint benchmark scoring. In some examples, in checkpoint benchmark scoring, the ML engine 320 produces a benchmark score for one or more checkpoint(s) of one or more ML model(s) 325, and keeps the checkpoint(s) that have the best (e.g., highest or lowest) benchmark scores in the ensemble. In some examples, if a new checkpoint is worse than an older checkpoint, the ML engine 320 can revert to the older checkpoint. The benchmark score for a checkpoint can represent a level of accuracy of the checkpoint and/or number of errors (e.g., false positive or false negative) by the checkpoint during the testing (e.g., against the testing data and/or the validation data). In some examples, an ensemble of the ML model(s) 325 can include multiple checkpoints of the same ML model.

In some examples, the system can intentionally generate benchmark questions that have no correct response. Such benchmark questions can be used to evaluate how a learner selects an answer when no option is correct, such as whether the learner tends to select a particular answer position, the longest response option, the shortest response option, or another answer-choice pattern. The benchmark response data, together with contextual data such as response-option length, answer order, topic, difficulty, and prior learner history, can be processed by an artificial intelligence (AI) model, a machine learning (ML) model, a statistical model, or a rule-based model to characterize learner-response patterns, adjust answer-order assignment, calibrate confidence estimates, personalize remediation, or optimize a learning experience. The benchmark questions can also be referred to as bias-detection items, answer-pattern probes, no-correct-answer items, calibration items, or learner-response probes.

In some examples, the ML model(s) 325 can be trained and/or updated (e.g., with training data 375 and/or the update 370) over a number of epochs, where each epoch includes one or more batches of training data. A batch refers to a subset of the training data that is processed together in a single forward and backward pass during model training. Processing data in batches can improve computational efficiency and stabilize gradient updates. As training progresses across multiple epochs, the model parameters are iteratively refined, enabling the model to generalize better to unseen data. The use of multiple epochs and batch-based updates allows the model to progressively learn complex patterns and reduce prediction error, thereby improving accuracy and/or robustness of the ML model(s) 325 over time. The number of epochs and batch size may be predetermined or dynamically adjusted based on performance of the ML model(s) 325, convergence criteria, and/or available computational resources.

In some examples, the ML model(s) 325 can be modified, either through the initial training (with the training data 375), an update 370 based on the feedback 365, or another modification to introduce randomness, variability, and/or uncertainty into an ensemble of the ML model(s) 325. In some examples, such modification(s) to the ML model(s) 325 can include dropout (e.g., Monte Carlo dropout), in which one or more weights or connections are selected at random and removed. In some examples, dropout can also be performed during inference, for instance to modify the output(s) 330 generated by the ML model(s) 325. The term Bayesian Machine Learning (BML) can refer to random dropout, random initialization, and/or other randomization-based modifications to the ML model(s) 325. In some examples, the modification(s) to the ML model(s) 325 can include a hyperparameter search and/or adjustment of hyperparameters. The hyperparameter search can involve training and/or updating different ML model(s) 325 with different values for hyperparameters and evaluating the relative performance of the ML model(s) 325 (e.g., against testing data and/or validation data where the correct output(s) are known) to identify which of the ML model(s) 325 performs best. Hyperparameters can include, for instance, temperature (e.g., influencing level creativity and/or randomness), top P (e.g., influencing level creativity and/or randomness), frequency penalty (e.g., to prevent repetitive language between one of the output(s) 330 and another), presence penalty (e.g., to encourage the ML model(s) 325 to introduce new data in the output(s) 330), other parameters or settings, or a combination thereof. The different hyperparameters can be modified to improve accuracy and reduce hallucinations (e.g., by reducing creativity or randomness, and/or reducing penalties for frequency or presence), to improve creativity (e.g., by increasing creativity or randomness, and/or increasing or decreasing penalties for frequency or presence), or a combination thereof.

In some examples, the ML model(s) 325 can be trained and/or updated (e.g., with training data 375 and/or update 370) using supervised learning, unsupervised learning, semi-supervised learning, reinforcement learning, self-supervised learning, transfer learning, and/or optimization algorithms such as gradient descent and its variants (e.g., stochastic gradient descent (SGD) or adaptive moment estimation (Adam)). These techniques can use labeled data, unlabeled data, generated supervisory signals, cumulative rewards, pre-trained models, and/or iterative parameter adjustment to improve the ML model(s) 325 for classification, regression, clustering, dimensionality reduction, sequential decision-making, and/or other tasks.

In some examples, the ML engine 320 can perform retrieval-augmented generation (RAG) using the model(s) 325. For instance, in some examples, the ML engine 320 can pre-process the input(s) 305 by retrieving additional information from one or more data store(s) 380 (e.g., any of the databases and/or other data structures discussed herein) and using the additional information to enhance the input(s) 305 before the input(s) 305 are processed by the ML model(s) 325 to generate the output(s) 330. For instance, in some examples, the enhanced versions of the input(s) 305 can include the additional information that the ML engine 320 retrieved from the one or more data store(s) 380. In some examples, the artificial intelligence system 300 can retrieve the additional information from one or more data store(s) 380 by querying the data store(s) 380 using RAG query(s) 350 generated by the ML model(s) 325 (or extracted from the input(s) 305 using the ML model(s) 325). In some examples, this RAG process provides the ML model(s) 325 with more relevant information, allowing the ML model(s) 325 to generate more accurate and/or personalized output(s) 330.

In some examples, the analyses and data processing operations discussed as being performed by the ML model(s) 325 can be performed by different types of AI algorithm(s) (other than ML model(s)) in addition to, or instead of, being performed by the ML model(s) 325. For instance, in some examples, AI algorithm(s) can be used to process the input(s) 305 (e.g., the information 310 and/or the previous output(s) 315) to generate the output(s) 330 (e.g., the enriched prompt 332, structured object 334, validation(s) 336, explanation(s) 338, score(s) 340, RAG query(s) 350, and/or tool command(s) 355). Examples of AI algorithm(s) that the AI system 300 can use in this way can include rule-based AI systems, expert-based AI systems, heuristic-based AI systems, genetic AI algorithms, search algorithms, symbolic AI algorithms, or combinations thereof.

In some examples, the input(s) 305 are received as a data stream, with a high volume of data continuing to be received and processed through the ML model(s) 325 over time. For instance, in some examples, the input(s) 305 can include assessment-generation requests, student-performance records, question-bank records, clinical-parameter records, media items, reference documents, validation records, feedback records, and/or user-interface inputs received across numerous users, cohorts, accounts, devices, and/or institutions. In some cases, the input(s) 305 received and processed can include hundreds, thousands, tens of thousands, hundreds of thousands, millions, tens of millions, hundreds of millions, or even billions of records per second, per minute, per hour, or the like. Furthermore, in some examples, the AI system 300 may be given a limited amount of time from receipt of given input(s) 305 to generate certain output(s) 330, such as the enriched prompt 332, structured object 334, validation(s) 336, explanation(s) 338, score(s) 340, RAG query(s) 350, and/or tool command(s) 355. These volumes of data can be impossible for human beings to process in such short timeframes. The ML model(s) 325 can thus provide a technical improvement by being able to process high volumes of data within short timeframes.

In some examples, the AI system 300 can extract batches 390 of data from the input(s) 305 (e.g., data stream) dynamically and in real-time (or near-real-time) as the data from the input(s) 305 continue to be received by the AI system 300 over time. In some examples, the AI system 300 can process the batches 390 of data (e.g., using the ML model(s) 325) dynamically and in real-time (or near-real-time) as the data from the input(s) 305 continue to be received by the AI system 300 to generate updates 395 to one or more outputs of the output(s) 330.

For instance, each batch 390 can be processed by the ML model(s) 325 to generate an update 395. The AI system 300 (e.g., the ML engine 320 and/or ML model(s) 325) can update previous output(s) 330 based on newer batches 390 sequentially, in parallel, after a threshold number of batches is received, or a combination thereof. The updates 395 can include feedback 365, training data, fine-tuning data, context data, and/or model parameters for training, re-training, fine-tuning, and/or updating the ML model(s) 325, in addition to or instead of updating the output(s) 330.

In some examples, one or more functional subsystems may be implemented as part of the components shown in the AI system 300 without being separately illustrated. For example, the ML engine 320, data store(s) 380, update 370, output(s) 330, and/or feedback engine(s) 360 may implement pre-processing and feature-engineering; embedding generation, indexing, and re-ranking; model registry, checkpoint, and deployment management; and/or policy and guardrail functions such as access control, retrieval filtering, structured output validation, tool-command validation, constrained decoding, permission checking for tool(s) 385, redaction, and escalation routing. The policy and guardrail functions can also include source-jurisdiction filtering, specialty filtering, allowed-tool routing, model-provider allowlists, prompt-length limits, and response-schema enforcement.

The architecture of the AI system 300 provides technical improvements by transforming and structuring input data into computational representations suitable for efficient inference, using indexed retrieval and caching to reduce repeated searches and network round trips, optimizing inference execution through batching, hardware-aware scheduling, quantization, distillation, and/or other model optimization techniques, and improving runtime reliability through calibrated uncertainty estimation, abstention/fallback routing, drift monitoring, validated tool invocation, tool-result feedback, and checkpoint-based rollback. These mechanisms can reduce inference latency, increase throughput under high-volume streams, reduce memory bandwidth consumption, reduce recomputation and retries, and improve accuracy and/or calibration under changing data conditions. By constraining model outputs to validated schemas and routing uncertain outputs through fallback paths, the AI system 300 can reduce downstream renderer failures, malformed database writes, and repeated model retries.

The AI system 300 can assign a unique generation-session identifier to each generation session. The unique generation-session identifier can be propagated through input(s) 305, previous output(s) 315, output(s) 330, RAG query(s) 350, tool command(s) 355, feedback 365, update 370, training data 375, data store(s) 380, tool(s) 385, batches 390, and updates 395. When a model response, tool result, validator result, or reference-verification result is received, the AI system 300 can compare an identifier associated with the received data against a current generation-session identifier and discard or archive stale data associated with a canceled, superseded, or different generation session. This prevents delayed model outputs from contaminating a later generation session. The generation-session identifier can function as a transaction identifier, session token, workflow identifier, request lineage identifier, or stale-output guard.

The AI system 300 can include a request-deduplication cache. Before transmitting a model request or tool command, the ML engine 320 can generate a content hash from request data, such as model identifier, prompt text, messages, model parameters, tool schema, referenced context, and generation-session data. If the content hash matches a stored request within a configurable time window, the ML engine 320 can reuse a cached response, avoid a duplicate API call, and store an audit event indicating that the cached response was used. The request-deduplication cache can reduce network traffic, reduce latency, and reduce computational load during validation loops that would otherwise submit repeated or substantially identical requests. The content hash can be generated after canonicalizing whitespace, ordering structured fields, normalizing model parameters, and removing volatile metadata so that semantically identical requests can map to the same cache entry.

The AI system 300 can support cancellation-aware processing. A cancellation state can be stored in a generation-status store and checked before model calls, before tool calls, before local GPU verification, before URL fetches, and before processing batches 390. When a cancellation state is detected, the ML engine 320 can stop scheduling additional work, close or cancel in-flight provider requests using provider-specific cancellation interfaces when available, release locks, update the generation-status store, and skip GPU verification for abandoned work. These operations reduce wasted accelerator cycles, reduce pending network calls, and prevent stale output(s) 330 from entering the data store(s) 380. Cancellation-aware processing can also release accelerator memory, close network sockets, clear queued tool commands, and prevent partial validation results from being written to an active assessment object.

The AI system 300 can isolate administrator-triggered generation from API-triggered generation. For example, a reentrant lock, distributed lock, database lock, or queue partition can prevent simultaneous generation sessions from sharing mutable prompt state, model-output state, or generation-status files. Separate status records can be maintained for administrator generation and API generation. The lock and status records can be released or updated when a session completes, fails, or is canceled, thereby reducing race conditions and cross-session contamination.

The AI system 300 can support multiple operating modes. In a first mode, the AI system 300 can perform generation and validation using remote models and model-provided tools. In a second mode, the AI system 300 can add local verification, programmatic length checks, local reference verification, or local deterministic checks i.e. length of answer choices In a testing mode, selected validator calls can return approval data while the remainder of the pipeline, including generation, metadata tracking, storage, image generation, explanation generation, and audit logging, continues to execute through the same data path used outside the testing mode. The local verification, programmatic length checks, and deterministic checks can be enabled or disabled by a configuration record, and in some operating modes the AI system 300 can rely on a generation model's self-validation output instead of separate validator-model calls. The operating modes can also be referred to as deployment modes, verification modes, validation modes, testing modes, fallback modes, or local-hybrid modes.

Another mode of operation is a revalidation mode in which an assessment object, explanation object, media object, or other generated content object is reinjected into validator models with or without associated mathematical-verification records, reference-verification records, source-material records, and feedback records. In the revalidation mode, the AI system 300 can acquire new evidence, new references, new web-search results, source material obtained directly from a model, updated audio material, updated video material, updated image material, translated content, new calculations, or other new data and check the object against current support. For example, when a newer ML model, updated guideline, newer journal article, or changed reference-source constraint becomes available, the AI system 300 can revalidate an existing assessment object and regenerate the assessment object, explanation, citation record, or media object using the newer model or reference material. Revalidation can be performed on a schedule, in response to a reference update, in response to model-registry changes, in response to user feedback, or before a stored assessment object is reused in a new quiz.

The same generation, validation, regeneration, and feedback-loop techniques can be applied to text, images, audio, video, translated content, and multimodal content. For example, a generated image, generated video, generated audio item, translated explanation, or translated question can be validated by one or more validator models, and validator feedback can be provided to a generating or refining model to change the corresponding content object.

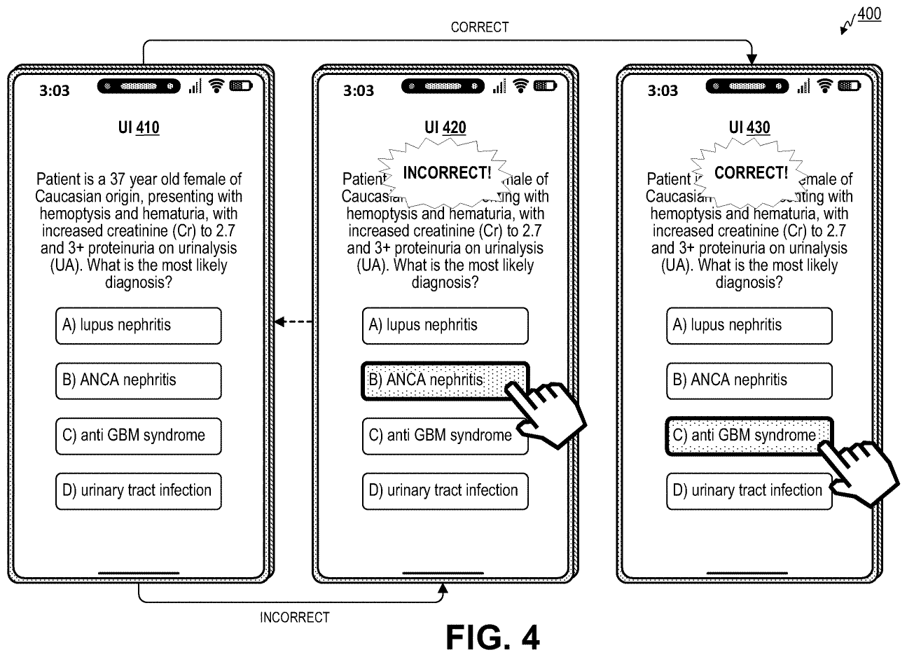

FIG. 4 is a conceptual diagram illustrating interactive user interfaces (UIs) for presenting assessment objects and receiving inputs selecting response-option objects. The user interfaces can be presented on an interface device or user device, such as one of the interface device(s) 210. The user interfaces can be web-based interfaces that the interface device(s) 210 access via a browser or other network-connected application, or can be app-based interfaces that the interface device(s) 210 access via an application that is locally-installed and/or locally-running on the interface device(s) 210. The user interfaces include a UI 410 that includes a question and multiple response options, a UI 420 in which an incorrect response option was selected, and a UI 430 in which a correct response option was selected. The user interface 410 can display an assessment object before a response is received and scored. In the illustrated example, the assessment object includes an assessment content portion that presents a medical vignette for a 37-year-old female of Caucasian origin with hemoptysis, hematuria, increased creatinine of 2.7, and 3+ proteinuria on urinalysis, followed by the question “What is the most likely diagnosis?” The user interface 410 can also display multiple response-option objects, such as lupus nephritis, anti-neutrophil cytoplasmic antibody (ANCA) nephritis, anti-glomerular basement membrane (anti-GBM) syndrome, and urinary tract infection. In some examples, these displayed response labels are generated after validation by the symbol assignment and watermarking engine 140, even though the underlying response-option objects were generated without user-facing labels.

A user interface 420 can represent an incorrect-answer state of the interactive assessment interface 400. For example, the user interface 420 can receive an input selecting a response-option object that does not correspond to the correct response-option object, and can output an incorrect status, an explanation, a prompt to review a concept, a score update, or another feedback item. A user interface 430 can represent a correct-answer state of the interactive assessment interface 400. For example, the user interface 430 can receive an input selecting the response-option object for anti-GBM syndrome and can output a correct status, an explanation, a score update, a progression event, or another feedback item. In some examples, each of the user interfaces 410, 420, and 430 can be displayed on one of the interface device(s) 210 and can provide input data for score generation, performance tracking, personalization, model updating, or feedback generation.

In another illustrative example, the user interface 410 can display an assessment object having an assessment content portion that states: “A 23-year-old African American female presents with joint pain, malar rash, increased creatinine to 1.5, and 3+ proteinuria on urinalysis. What is the most likely diagnosis?” The corresponding response-option objects can include lupus nephritis, ANCA nephritis, urinary tract infection, and anti-GBM syndrome. In this example, selection of lupus nephritis can correspond to a correct response-option object, while selection of one of the other response-option objects can correspond to an incorrect response-option object.

As a further illustrative example, the user interface 410 can display an assessment object generated from an enriched prompt that includes patient age and origin. The assessment content portion can state: “A 17-year-old female of Chinese origin presents with joint pain, malar rash, increased creatinine to 1.7, and 1+ proteinuria on urinalysis. What is the most likely diagnosis?” The response-option objects can include lupus nephritis, ANCA nephritis, anti-GBM syndrome, and urinary tract infection. This example illustrates that a change in the enriched prompt can change the assessment content portion while preserving a comparable response-option structure for presentation through the interactive assessment interface 400.

As yet another illustrative example, the user interface 410 can display an assessment object generated from an enriched prompt that includes age, sex, medical history, and medication context. The assessment content portion can state: “A 76-year-old Caucasian male presents with joint pain, malar rash, increased creatinine to 1.7, and 1+ proteinuria on urinalysis. His past medical history is positive for hypertension and diabetes. His medications include insulin, hydralazine, and amlodipine. What is the most likely diagnosis?” The response-option objects can include drug-induced lupus nephritis, ANCA nephritis, anti-GBM syndrome, and diabetic nephropathy. In this example, the response-option object for drug-induced lupus nephritis can be the correct response-option object, and the other response-option objects can be distractor response-option objects.

In some examples, a draft assessment object can include a question stem or other assessment content portion that unintentionally reveals the correct answer, such as by stating that presented findings are “typical for lupus nephritis” before asking for the most likely diagnosis. The validator model 130, validation(s) 336, and/or process 500 can reject or flag such an assessment object before the assessment object is displayed in the user interface 410, or can cause the generation model 120 to regenerate the assessment content portion so that the user interface 410 presents the response-option objects without answer-revealing language.

The interactive assessment interface 400 can present an assessment object in text, audio, image, video, three-dimensional, augmented-reality, virtual-reality, mixed-reality, game, flashcard, poll, document, or simulation form. For example, the system can convert the assessment content portion and response-option objects into first-person patient audio, second-person learner prompts, clinician-to-learner dialog, generated video, animated video, a three-dimensional rendered patient, a clinical-room simulation, a mobile infographic, a square social-media infographic, or a question-of-the-day or question-of-the-week interface. The same structured assessment object can be rendered in more than one modality, so that a text question can be converted into audio, video, animated dialog, a flashcard, a simulation prompt, or another user-facing format while retaining the same underlying response-option objects and scoring fields. Because the interface can render multiple modalities from the same structured object, the system can keep scoring, validation, reference tracking, and watermarking consistent across text, audio, video, and simulation presentations.

The interactive assessment interface 400 can receive inputs through monitors, touchscreens, microphones, speakers, cameras, virtual-reality (VR) headsets, augmented-reality (AR) glasses, haptic gloves, controllers, manipulators, wearable devices, physiological sensors, EEG sensors, electromagnetic-field sensors, implantable sensors, implantable leads, external electrodes, or other input/output hardware. In some examples, the interface 400 can use sensor data to determine attention, distraction, confidence, stress, anxiety, recall state, memorization state, preferred modality, or readiness for a subsequent assessment object. The system can use the determined state to select a next question, adjust difficulty, select a media modality, repeat content, generate remediation, or update a study path.

The input/output hardware, sensors, leads, electrodes, haptic actuators, displays, speakers, and stimulation interfaces can participate in biofeedback loops and learning-experience optimization. For example, the system can use progress, performance, EEG data, vital signs, video recognition, audio recognition, eye tracking, pupil tracking, skin-moisture data, blood-pressure data, oxygen-saturation data, age, language, and other authorized signals to select or generate assessment content, determine when to provide reinforcement, and support memorization or review. In examples involving stimulation of reward-related circuits, cortex, hippocampus, or other neurological structures, the system can store permission data, safety limits, stimulation parameters, and audit records. In some examples, the system can select different types of content (e.g., different types of questions and/or responses) based on the user's mood, stress level, tiredness level, recent activity, level of education, area of expertise, or a combination thereof. In an illustrative example, when sensor data indicates the user has elevated stress or arousal, the system can select more intense fields such as emergency-medicine content-whereas when sensor data indicates the user has lower levels of stress or arousal (e.g., relaxation), the system can select lower-intensity review content such as plastic surgery. In some examples, when sensor data indicates relaxation, the system can schedule sleep-state review cues when authorized sensor data indicates an appropriate sleep stage. Biofeedback or stimulation outputs can be used as reinforcement, pacing signals, recall cues, or reward-associated signals under stored permission data and safety limits. The permission data and safety limits can include user consent records, device-pairing records, stimulation limits, session duration limits, clinician or administrator authorization records, and audit events.

In some examples, the interface 400 can support ambient retrieval of previously studied content. For instance, a learner device can receive a voice query asking for a reminder of key facts studied during a prior session or can analyze authorized audio or video from a learner-patient or learner-instructor interaction to retrieve assessment content, explanations, or references that are relevant to the interaction. The retrieved content can be presented at a point of care, during study, during review, or during a later remediation session.

The interface 400 can also receive feedback about a question, response-option object, explanation, citation, image, audio item, video item, simulation, or other media content. The feedback can be used to deactivate an assessment object, reactivate a previously deactivated assessment object, request revalidation using a newer model or models, update source references, revalidate mathematical calculations, update a training dataset, adjust a user-specific study path, retrieve a cached assessment object, or generate a new assessment object. Feedback analysis can consider a user performance level, percentile, category history, social sharing history, prior feedback history, paid-access status, or other account metadata.

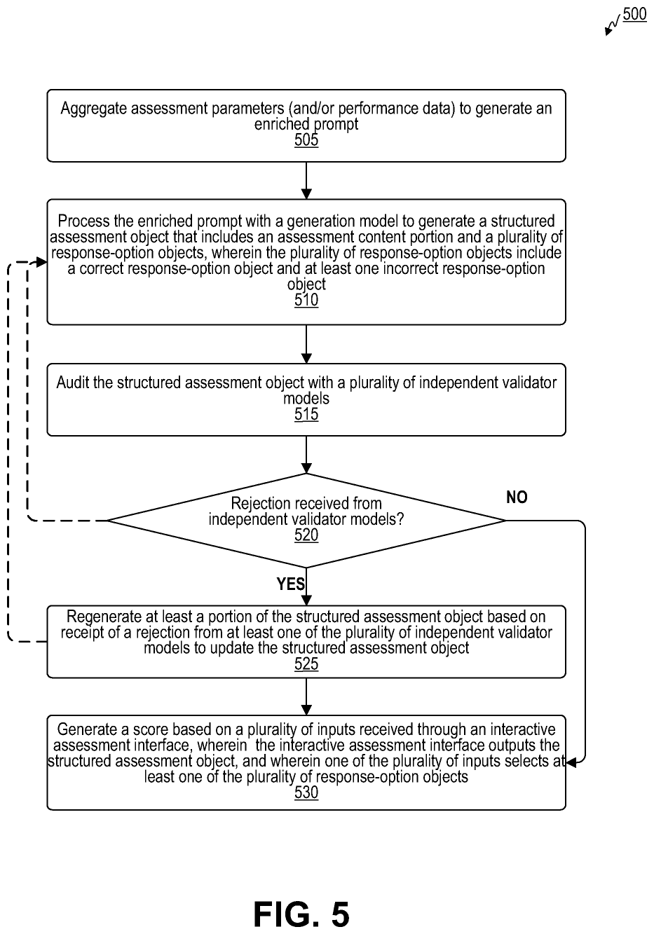

FIG. 5 is a flow diagram illustrating a process 500 for multi-model assessment generation and validation. The process 500 can be performed by, and/or using, an assessment generation and validation system. The assessment generation and validation system can include, for instance, the system 100, the student database 115, the enriched prompt engine 110, the generation model 120, the validator model 130, the mathematical verification model 135, the symbol assignment and watermarking engine 140, the reference tracker 150, the explanation engine 155, the system 200, the interface device(s) 210, the server(s) 215, the ML engine 220, the ML model(s) 225, the data store system(s) 230, the AI system 300, the ML engine 320, the ML model(s) 325, the feedback engine(s) 360, the data store(s) 380, the tool(s) 385, an interface device that displays the user interfaces of FIG. 4 (e.g., UI 410, UI 420, UI 430), the computing system 600, an apparatus, a medical device, a terminal, a processor that performs instructions stored in a non-transitory computer-readable storage medium, any subsystems or components of any of the above-listed systems, any other computing systems disclosed herein, or a combination thereof.

In some examples, the process 500 retrieves an already-created structured assessment object from a cache, question bank, reference-indexed store, or other data store instead of generating a new structured assessment object. The retrieved structured assessment object can be selected based on a current learning path, topic, category, difficulty, learner history, teacher preference, prior correct responses, prior incorrect responses, time since last exposure, desired spacing interval, or a model-generated recommendation. The retrieved structured assessment object can be revalidated before presentation when a revalidation trigger is satisfied. The retrieved structured assessment object can be mixed with newly generated structured assessment objects in the same quiz, study set, remediation set, or batch, and the proportion of retrieved and newly generated objects can be selected based on freshness, coverage, learner history, or available computing resources. For example, a remediation session can include stored cardiology items that were previously validated, newly generated items directed to recent incorrect answers, and benchmark items that evaluate answer-selection bias. In some examples, a retrieved or newly generated structured assessment object can be selected from, or generated using, simulated patient records stored in the data store(s) 380 as training data 375, prompt context, validation context, or personalization context. The simulated patient records can be generated without exposing corresponding real-patient identifiers and can be used to provide coverage for rare clinical presentations, underrepresented feature combinations, or newly added outcome categories.

At operation 505, the assessment generation and validation system (or a subsystem thereof) is configured to, and can, aggregate assessment parameters and, when available, performance data to generate an enriched prompt. The enriched prompt can correspond to the enriched prompt 118 generated by the enriched prompt engine 110 and/or the enriched prompt 332 included among the output(s) 330 of the AI system 300. In some examples, performance data comprises student performance metrics stored in the student database 115, the data store system(s) 230, and/or the data store(s) 380. In other examples, such as for a new user or a user with insufficient history, the enriched prompt can be generated using default data, cohort-level data, teacher-selected parameters, curriculum parameters, assessment parameters, or clinical parameters without requiring user-specific performance data. The assessment parameters can comprise clinical parameters 111 and/or question parameters 113, including at least one of a patient age, a patient sex, one or more vital signs, a medical history, a category, a difficulty, a question type, or a case type. The enriched prompt can also include pedagogical context 117, media content 119, retrieved information obtained through RAG query(s) 350, and tool results obtained through tool command(s) 355. In some examples, the assessment generation and validation system (or a subsystem thereof) is configured to, and can, generate simulated patient data by receiving or generating a source seed that includes feature parameters and outcome records. The feature parameters can identify possible values, ranges, minimum thresholds, maximum thresholds, distributions, categorical values, missing-value rules, or combinations thereof for features used in a clinical vignette or assessment object. The outcome records can identify diagnoses, recommended tests, recommended treatments, outcome likelihoods, recommendation strengths, feature-importance values, teaching points, or combinations thereof. The system can then sample, semi-randomly select, or deterministically assign feature values according to the feature parameters to generate multiple simulated patient records associated with the outcome records. For example, the system can generate simulated records having age values selected from a specified age range, body mass index values sampled from a Gaussian distribution, symptom-severity values selected from categorical levels, and laboratory values selected from outcome-specific ranges. The generated simulated patient data can include physical characteristics, demographic values, medical history, medication history, allergy history, vital signs, symptoms, physical-examination findings, laboratory results, imaging findings, exposure history, severity values, missing-value indicators, and metadata. The simulated patient data can be stored as tables, database rows, structured objects, tensors, prompt-context records, or source-seed-linked records, and can be used by the enriched prompt engine 110, ML engine 320, ML model(s) 325, feedback engine(s) 360, or data store(s) 380.

In some examples, during presentation of an assessment object, a learner device can read aloud a question or explanation, receive a spoken response through a microphone, capture local interaction data, and execute a local model to analyze speech, audio quality, confidence, hesitation, pronunciation, reasoning, or another signal. The local model output can be used as input to score generation, status generation, remediation, next-question selection, study-path updating, or later assessment-object generation.

At operation 510, the assessment generation and validation system (or a subsystem thereof) is configured to, and can, process the enriched prompt with a generation model to generate or refine a structured assessment object that includes an assessment content portion and a plurality of response-option objects. The generation model can include the generation model 120, the ML model(s) 225, and/or the ML model(s) 325. The structured assessment object can correspond to the structured object 125 and/or the structured object 334. In some examples, the structured assessment object is a JavaScript Object Notation object. In some examples, the assessment content portion comprises a question stem, such as a question displayed in UI 410. In some examples, the assessment content portion comprises a clinical vignette, patient scenario, laboratory-data set, image-based prompt, audio prompt, video prompt, simulation state, or another assessment prompt. The assessment content portion can also be referred to as question content, prompt content, item content, stem content, vignette content, scenario data, case data, or learner-facing content.

At operation 510, the plurality of response-option objects include a correct response-option object and at least one incorrect response-option object. The generation model can generate the correct response-option object and the at least one incorrect response-option object together in a single pass, or can generate the response-option objects in multiple passes. For example, the generation model 120 can use a correct response-option object from a current question, prior question, related clinical vignette, related category, or previously generated structured object as context for generating a distractor having a target similarity to the correct response-option object. The response-option objects can be displayed as selectable options in UI 410, UI 420, and/or UI 430 after approval and label assignment. In some examples, the difficulty level controls a similarity between the correct response-option object and the at least one incorrect response-option object, such as by controlling semantic similarity, length similarity, numerical closeness, overlap in clinical findings, or a number of implicit reasoning steps. The response-option objects can also be generated in parallel model calls, such as by sending separate prompts for the correct response-option object and each distractor response-option object to one or more generation models. In some examples, a first generation model generates the assessment content portion, a second generation model generates the correct response-option object, and one or more additional generation models generate distractor response-option objects. The target similarity can be expressed as a semantic-similarity score, embedding distance, concept-overlap score, length-difference value, numerical-distance value, or clinical-plausibility score.

At operation 515, the assessment generation and validation system (or a subsystem thereof) is configured to, and can, audit the structured assessment object with a plurality of independent validator models. The independent validator models can include instances of the validator model 130, the mathematical verification model 135, the ML model(s) 225, and/or the ML model(s) 325. In some examples, the plurality of independent validator models comprises models from different model providers, different model families, different model sizes, different deployment locations, or different validation roles. In some examples, auditing the structured assessment object comprises medical fact-checking, mathematical verification, temporal verification, logical verification, citation verification, formatting verification, language review, policy review, or combinations thereof. In some examples, the plurality of independent validator models are restricted to comment-only permissions with respect to the structured assessment object, so that the validator models generate validation(s) 336 without directly modifying the structured assessment object.

At decision point 520, if the audit results in a rejection received from at least one of the independent validator models, then the process 500 proceeds from operation 515 to operation 525 and can return to operation 510 after validator feedback is incorporated. At decision point 520, if the audit does not result in any rejection received from at least one of the independent validator models, such as when a consensus approval condition is satisfied, then the process 500 proceeds from operation 515 to operation 530. The rejection can include a rejection status, a reason, validator-model identity, citation-verification result, mathematical-verification result, temporal-consistency result, logical-consistency result, quality-metric result, reference-location request, model-to-model communication record, or suggested correction. In some examples, the rejection can include feedback, for instance including requested changes-with those changes then made at operation 525.

At operation 525, the assessment generation and validation system (or a subsystem thereof) is configured to, and can, regenerate at least a portion of the structured assessment object based on receipt of a rejection from at least one of the plurality of independent validator models to generate an updated structured assessment object. The regeneration can be performed by providing the rejection status and associated validator feedback to the generation model 120 through the feedback loop 190 and/or as previous output(s) 315 to the ML model(s) 325. The regenerated portion can include a question stem, clinical vignette, response-option object, explanation, citation, numerical value, metadata field, media item, or another portion of the structured assessment object.

In some examples, the process 500 can return to operation 510 after decision point 520 and/or operation 525, so that at least the portion of the structured assessment object is regenerated using the enriched prompt of operation 510 as well as feedback associated with the rejection(s) (of decision point 520) and/or feedback associated with a regeneration process (of operation 525).

In some examples, the assessment generation and validation system (or a subsystem thereof) is configured to, and can, repeat the auditing and regenerating until a consensus approval condition or quality-metric threshold is satisfied. In some examples, the consensus approval condition is based on approval statuses from each of the plurality of independent validator models. The consensus approval condition can also be based on approval by a threshold number of validator models, approval by specified validator types, successful deterministic verification by the mathematical verification model 135, successful citation verification by the reference tracker 150, satisfaction of reference-source criteria, satisfaction of answer-choice length rules, satisfaction of semantic-distance rules, or combinations thereof. The threshold number of validator models can be expressed as an absolute count, a fraction, a percentage, or a role-specific rule, such as approval by at least two of three validator models or approval by at least one medical validator and one citation validator. During repeated auditing and regeneration, assessment objects, response-option objects, explanations, or references that have already satisfied the applicable approval condition can be removed from an active batch and stored in an approved-item set while remaining unresolved objects continue through the loop. The approved-item set can be stored as a locked set, frozen set, accepted subset, approved working set, or inactive batch subset that is omitted from later regeneration prompts.

In some examples, the assessment generation and validation system (or a subsystem thereof) is configured to, and can, assign randomized user-facing response labels to the response-option objects after the structured assessment object satisfies an approval condition. The randomized user-facing response labels can be assigned by the symbol assignment and watermarking engine 140, and can correspond to labels displayed with selectable response-option objects in UI 410, UI 420, and/or UI 430. Deferring randomized label assignment until after validation can maintain label-agnostic generation and can reduce leakage of a correct label into the assessment content portion, explanation(s) 338, validation(s) 336, or audit records.

In some examples, the assessment generation and validation system (or a subsystem thereof) is configured to, and can, store a mapping between a user identifier, an assessment identifier, and a correct response label. The mapping can be stored by the symbol assignment and watermarking engine 140, the reference tracker 150, the student database 115, the data store system(s) 230, and/or the data store(s) 380. The mapping can support randomized presentation, watermarking, forensic tracing, score generation, and reconstruction of a version of the structured assessment object that was displayed to a particular user.

At operation 530, the assessment generation and validation system (or a subsystem thereof) is configured to, and can, generate a score or status based on a plurality of inputs received through an interactive assessment interface. The interactive assessment interface can output the structured assessment object using the interface 400, UI 410, UI 420, UI 430, interface device(s) 210, output device 635, input device 645, or combinations thereof. One of the plurality of inputs can select at least one of the plurality of response-option objects. The score or status of operation 530 can include the score(s) 340, an item score, assessment score, pass/fail status, accepted/rejected status, approval count, validator-approval count, confidence score, or another metric. For example, the score can be based on a number of selections of correct responses to questions, such as selections received through UI 430, and/or a number of selections of incorrect responses to questions, such as selections received through UI 420. The score or status can update a student-performance record, a difficulty estimate, a recommendation for a next assessment object, a query for retrieving an existing assessment object, a generation request for many next assessment objects, a feedback record, a model-training dataset, or a forensic audit record. In some examples, the score or status of operation 530 can be used as feedback 365 to update reputation scores, sampling weights, or reliability scores associated with an expert source, source seed, simulated patient population, simulated patient record, validator model, or querying user. Based on the updated reputation scores, sampling weights, or reliability scores, the assessment generation and validation system can regenerate, resample, reweight, or exclude simulated patient data used as training data 375 or prompt context.

In some examples, operation 530 can trigger one or more proactive actions while a quiz, test, examination, practice set, remediation set, or other assessment is still in progress. The proactive actions can be based on a running grade, score so far, streak of correct responses, streak of incorrect responses, response latency, answer-change frequency, confidence value, sensor signal, or combination thereof. When the score or status indicates that the user is answering more than a threshold number or percentage of questions incorrectly, the assessment generation and validation system can proactively and/or dynamically reduce difficulty for one or more remaining assessment objects. Reducing difficulty can include selecting easier stored assessment objects, generating easier new assessment objects, reducing a number of implicit reasoning steps, reducing a number of irrelevant findings, adding hints, increasing explanation detail, increasing time limits, or simplifying media content. Reducing difficulty can also include modifying only unpresented response-option objects so that incorrect response-option objects are more clearly distinguishable from the correct response-option object, while preserving the approved question stem or clinical vignette. When the score or status indicates that the user is answering more than a threshold number or percentage of questions correctly, the assessment generation and validation system can proactively and/or dynamically increase difficulty for one or more remaining assessment objects. Increasing difficulty can include selecting harder stored assessment objects, generating harder new assessment objects, increasing a number of implicit reasoning steps, adding plausible irrelevant findings, reducing hints, reducing time limits, increasing media complexity, or requiring finer distinctions among response-option objects. Increasing difficulty can also include modifying only unpresented response-option objects so that incorrect response-option objects are harder to distinguish from the correct response-option object while remaining incorrect under the validation(s) 336. In some examples, the proactive actions include regenerating, refining, validating, locking, or replacing only assessment objects that have not yet been presented through the interactive assessment interface, thereby avoiding changes to already-presented questions while preserving auditability. The assessment generation and validation system can associate the proactive actions with generation-session identifiers, content hashes, label mappings, validator results, and user-interface state so that late-arriving regenerated content is not presented out of order. In some examples, the proactive actions can be performed via tool command(s) 355 to tool(s) 385. In some examples, operation 530 can trigger one or more proactive actions after a quiz, test, examination, practice set, remediation set, or other assessment is complete. In some examples, proactive actions can include generating another test, providing a reward or encouragement if the user is scoring well, providing a punishment or encouragement if the user is scoring poorly, activating or enabling access by the user to a website, application, or other content, preventing or disabling access by the user to a website, application, or other content, scheduling a follow-up test (e.g., via tool command(s) 355 to tool(s) 385 such as a calendar or scheduling service) or a graduation or other next step, generating a certificate of completion, updating a user profile or user credentials (e.g., to indicate an update in what the user is qualified for), or a combination thereof. For example, the system can generate a new validated question set, update an access-control record, and transmit a calendar command in the same automated response to a running score without requiring a human administrator to manually rebuild the assessment while the assessment session remains active.

In some examples, the assessment generation and validation system (or a subsystem thereof) is configured to, and can, execute a deterministic verification program to verify a numerical value contained in the structured assessment object. The deterministic verification program can be executed by the mathematical verification model 135, tool(s) 385, processor 610, service 1 632, service 2 634, service 3 636, or another software or hardware service. In some examples, the numerical value includes at least one of a body mass index, a medication dose, a rate, a pressure, a weight, a volume, a mass, or an age. Additional numerical values can include an estimated glomerular filtration rate, creatinine clearance, anion gap, corrected calcium, dosage interval, date interval, ratio, or unit conversion. For example, the deterministic verification program can calculate a body mass index from mass and height values in a question stem, verify a medication dose from patient weight and renal-function values, or verify that a response-option object uses units consistent with the assessment content portion. The deterministic verification program can output a calculation trace, input-value table, unit-conversion record, formula identifier, rounding rule, and pass/fail status for storage with the structured assessment object.

In some examples, the assessment generation and validation system (or a subsystem thereof) is configured to, and can, store, in a reference tracker, a verification status and a validator model identity for a citation associated with the structured assessment object. The reference tracker can correspond to the reference tracker 150 and can store citation identifiers, reference uniform resource locators (URLs), source-document identifiers, passage identifiers, annotation identifiers, validation(s) 336, timestamps, model identities, provider identities, and mappings between portions of the structured assessment object and cited material. The reference tracker can identify whether a citation was proposed by the generation model 120, proposed by a validator model 130, retrieved by RAG query(s) 350, returned by tool(s) 385, or selected by a controlled update service after a validator model suggested a more accurate reference. The reference tracker can store generator-proposed sources, validator-proposed sources, replacement sources, rejected sources, and source-quality scores as separate records linked to the same citation field.

In some examples, the assessment generation and validation system (or a subsystem thereof) is configured to, and can, verify a reference associated with the structured assessment object by splitting fetched reference content into sentence-boundary sections and processing at least one of the sentence-boundary sections with a local verification model executing on local hardware. The local verification model can execute using processor 610, cache 612, memory 615, storage device 630, a graphics processing unit, a tensor processing unit, a neural processing unit, a field programmable gate array, or another accelerator. Sentence-boundary sectioning can allow a verification module to process a complete sentence for citation support while compressing, summarizing, or omitting less relevant sections to reduce context-window consumption, memory use, and network transmission.

In some examples, the assessment generation and validation system (or a subsystem thereof) is configured to, and can, associate a generation-session identifier with the structured assessment object and omit from active presentation a model output that is associated with a canceled or superseded generation-session identifier. The omitted model output can be discarded, archived, or stored in a rejected-output repository for debugging, future training, negative training, adversarial training, and/or quality analysis. The generation-session identifier can be propagated through the enriched prompt 118, structured object 125, output(s) 330, validation(s) 336, RAG query(s) 350, tool command(s) 355, feedback 365, data store(s) 380, batch records 390, and updates 395 so that asynchronous responses can be matched to the active generation session before being rendered, scored, or used for regeneration.

In some examples, the assessment generation and validation system (or a subsystem thereof) is configured to, and can, generate a content hash for a model request and reuse a cached model response when the content hash matches a previously stored content hash within a configurable time window. The content hash can be based on a normalized enriched prompt, model identifier, provider identifier, patient parameters, question parameters, tool-configuration record, reference-context record, role identifier, tenant identifier, or combinations thereof. Reusing the cached model response can reduce duplicate model calls, repeated tool calls, network traffic, and accelerator utilization for repeated or near-simultaneous requests. Different content hashes can be computed for a full object, a field, a prompt segment, a reference set, a validator request, or a tool command, allowing reuse at different granularities.

In some examples, the content hash can be based on the entirety of the structured assessment object, or on selected portions of the structured assessment object, and can be used as a version identifier, cache identifier, deduplication identifier, provenance identifier, or identifier for a particular assessment object.

In some examples, the assessment generation and validation system (or a subsystem thereof) is configured to, and can, select, using a model registry, a model for a pipeline role based on a declared capability of the model. The model registry can map roles such as generation, validation, explanation generation, reference verification, media generation, local verification, reranking, and scoring to model identifiers, provider identifiers, credentials or credential references, context-window limits, tool interfaces, rate limits, and declared capabilities. The declared capabilities can indicate support for web search, web fetch, code execution, local execution, image generation, audio generation, video generation, structured output, or other model or tool operations.

In some examples, the interactive assessment interface outputs at least one of audio content, video content, three-dimensional content, augmented-reality content, virtual-reality content, a flashcard, a poll, or a simulation. For example, the interface 400 can output a text question through UI 410, an explanation through UI 420 or UI 430, a first-person patient audio clip through output device 635, a video or three-dimensional patient simulation through interface device(s) 210, a flashcard or poll through a mobile interface, or an augmented-reality or virtual-reality scenario through a headset or other extended-reality device.

In some examples, the assessment generation and validation system (or a subsystem thereof) is configured to, and can, receive sensor data through the interactive assessment interface and adjust a difficulty, pacing, media modality, or next assessment object based on the sensor data. The sensor data can be received from interface device(s) 210, input device 645, a camera, microphone, eye-tracking sensor, gaze sensor, pupil sensor, haptic device, wearable device, physiological sensor, heart-rate sensor, blood-pressure sensor, oxygen-saturation sensor, skin-moisture sensor, electroencephalography (EEG) sensor, electromagnetic-field sensor, implantable sensor, external electrode, or combinations thereof. The sensor data can indicate attention, distraction, confidence, stress, anxiety, recall state, memorization state, modality preference, or readiness, and can be used with score(s) 340 and feedback 365 to select a subsequent assessment object. In some examples, the system can select different types of content (e.g., different types of questions and/or responses) based on the user's mood, stress level, tiredness level, recent activity, level of education, area of expertise, or a combination thereof. In an illustrative example, when sensor data indicates the user has elevated stress or arousal, the system can select more intense fields such as emergency-medicine content-whereas when sensor data indicates the user has lower levels of stress or arousal (e.g., relaxation), the system can select lower-intensity review content such as plastic surgery. In some examples, when sensor data indicates relaxation, the system can schedule sleep-state review cues when authorized sensor data indicates an appropriate sleep stage. Biofeedback or stimulation outputs can be used as reinforcement, pacing signals, recall cues, or reward-associated signals under stored permission data and safety limits.

In some examples, the assessment generation and validation system (or a subsystem thereof) is configured to, and can, generate a batch of structured assessment objects according to a batch context dictionary that specifies category quotas, question-type definitions, case-type definitions, and difficulty-specific requirements. The batch context dictionary can also specify per-question context records, category explanations, teaching points, allowed media types, reference requirements, target output counts, validation thresholds, and schedule data. The batch can correspond to batches 390, and updates to the batch can correspond to updates 395. The batch can be validated as a batch, so that shared reference material, shared category definitions, shared source filters, and shared validation thresholds are reused across multiple structured assessment objects. For example, five or six journal references can be retrieved once and used to generate and validate ten to twenty related medical questions, thereby reducing repeated retrieval, prompt-token usage, and citation-verification work. Batch validation can improve technical efficiency when related assessment objects share a specialty, reference set, source filter, difficulty level, validation rubric, or response-option quality rule.

In some examples, the assessment generation and validation system (or a subsystem thereof) is configured to, and can, store an annotation identifier for a citation associated with the structured assessment object. In some examples, the annotation identifier is based at least in part on a content hash. The annotation identifier can be generated from a counter, citation hash, passage hash, sentence hash, model-output hash, structured-object hash, generation-session identifier, or combinations thereof. The annotation identifier can be stored by the reference tracker 150 and can link a citation to a field of the structured object 125 or structured object 334.

In some examples, the assessment generation and validation system (or a subsystem thereof) is configured to, and can, store cancellation state in a generation-status store and check the cancellation state before scheduling a model call, tool call, or local verification operation. The generation-status store can be implemented using the student database 115, data store system(s) 230, data store(s) 380, storage device 630, memory 615, or another storage system. The cancellation state can be checked before invoking the generation model 120, validator model 130, mathematical verification model 135, ML model(s) 325, RAG query(s) 350, tool command(s) 355, tool(s) 385, or local hardware used for reference verification.

The process 500 can generate tags, keywords, social-media metadata, poll metadata, flashcards, document sections, books, Portable Document Format (PDF) files, word-processing files, slide decks, games, or other derivative content from an approved structured assessment object. For example, a tool command 355 can cause a coding model or game-generation tool to generate an educational game, cause a document-generation tool to generate a book or handout, cause a media-generation tool to generate images or video, or cause a social-media tool to generate handles, hashtags, and poll-format content. Derivative content can also include language translations, format translations, speech outputs, video outputs, synchronized lip-movement animations, true-or-false conversions, flashcard conversions, slide-deck conversions, or document-format conversions generated from the same approved structured assessment object.

Tags, keywords, difficulty values, topics, categories, case types, question types, learner-performance records, and content embeddings can be used to identify related assessment objects by content, difficulty, topic, category, or learning objective. One or more AI models, ML models, recommendation models, ranking models, statistical models, or rule-based selectors can use such data to recommend or retrieve a next assessment object during a learning path. A selector can use content matching, embedding similarity, prior-performance data, and topic filters to combine stored assessment objects with freshly generated assessment objects for a requested category, such as cardiology, endocrinology, nephrology, or another subject. In some examples, the proactive actions include generating another quiz, test, examination, practice set, remediation set, challenge set, flashcard set, study plan, micro-lesson, simulation, or follow-up assessment based on the score or status of operation 530. The proactive actions can include outputting a reward, encouragement, badge, certificate, token, score adjustment, content-unlock event, access-right update, positive reinforcement signal, or biofeedback reward when the score or status satisfies a performance threshold. The proactive actions can include outputting a punishment, corrective consequence, warning, prerequisite-review assignment, content-lock event, access restriction, additional remediation requirement, tutor-notification event, or encouragement when the score or status fails to satisfy a performance threshold. The proactive actions can include activating access by the user to a website, application, course module, media item, clinical simulation, laboratory module, game, document, or other content. The proactive actions can include preventing, disabling, pausing, or limiting access by the user to a website, application, course module, media item, clinical simulation, laboratory module, game, document, or other content. The proactive actions can include scheduling a follow-up quiz, test, examination, office-hours event, reminder, tutoring session, graduation event, credentialing step, course-enrollment step, clinical rotation step, or other next step by generating tool command(s) 355. The proactive actions can include sending a notification, message, email, calendar invitation, learning-management-system update, administrator alert, teacher alert, parent alert, clinician alert, or application-programming-interface request through tool command(s) 355 or communication interface 640. The proactive actions can include updating a learner profile, category mastery map, spaced-repetition schedule, cohort analytics record, access-control list, account state, payment or subscription state, credential record, certificate record, or audit log.

The process 500 can include periodic or event-triggered revalidation. Revalidation can be triggered by negative user feedback, a new model becoming available, an updated guideline, a changed citation-verification result, a drift metric, a threshold number of incorrect responses, or a scheduled review. Revalidation can update a verification status, deactivate an assessment object, reactivate an assessment object, regenerate a portion of the structured assessment object, update explanation text, seek new references, seek new audio material, seek new video material, seek new image material, perform new mathematical verification, or seek new evidence or technology while preserving an audit trail of prior versions. Rejected content can be retained as a bad-example record, negative training record, or quality-analysis record even when the rejected content is not presented to a learner. When newer model versions, newer references, updated guidelines, or improved validators become available, the revalidation can compare old and new validation results and selectively regenerate portions of stored assessment objects that are no longer sufficiently supported. A revalidation record can store the old validator identity, new validator identity, old reference set, new reference set, changed fields, and reason for revalidation so that the system can reconstruct why a stored object changed. In some examples, the assessment generation and validation system (or a subsystem thereof) is configured to, and can, invoke a simulated-patient-data generation service, a source-seed management service, a reputation-score service, a training-data reweighting service, a model-update evaluation service, or a staged-deployment service during process 500. The process 500 can generate candidate model updates, store measured performance results for the candidate model updates, train an auxiliary update-selection model from the measured performance results, and select a model update predicted to improve at least one processing characteristic. The processing characteristic can include accuracy, confidence, latency, memory use, power use, heat generation, false-positive rate, false-negative rate, validation-pass rate, calibration error, or another metric associated with generation, validation, scoring, retrieval, or presentation of assessment objects. In some examples, a feedback engine 360 executes a staged model-update workflow via feedback 365 and update 370 by evaluating a candidate model checkpoint against validation data, simulated clinical records, live feedback records, shadow-mode production records, or combinations thereof before the candidate model checkpoint is selected for active use. In distributed examples, a first computing system 600 can generate simulated patient records, a second computing system 600 can train or fine-tune ML model(s) 325 using the simulated patient records, and a third computing system 600 can present assessment objects or predicted outcomes generated by the trained or fine-tuned ML model(s) 325. In some examples, transformed, simulated, anonymized, redacted, or aggregated patient-context data is transmitted between computing systems while more sensitive source data remains in a local or permission-restricted environment.

The process 500 can provide a technical solution to a stale-output problem that can occur in asynchronous model pipelines. By propagating generation-session identifiers, checking cancellation state, and discarding outputs associated with canceled or superseded identifiers, the assessment generation and validation system can prevent a late-arriving model output, tool result, or local verification result from overwriting a current structured assessment object or being rendered through the interactive assessment interface. This can improve consistency of stored records, displayed content, and score generation while reducing unnecessary database writes and interface updates. This session-based gating can also reduce race conditions between administrator-triggered generation, application-programming-interface-triggered generation, batch regeneration, and user-triggered revalidation.

The process 500 can provide a technical solution to a data-integrity problem that can occur when generated natural-language content, structured response-option objects, citations, and numerical fields are produced by different models or tools. By maintaining the structured assessment object as a machine-readable object, restricting validator models to comment-only permissions, routing rejection data through operation 525, and storing reference and annotation metadata in the reference tracker 150, the assessment generation and validation system can preserve object provenance, reduce nondeterministic field mutation, and support reproducible regeneration of rejected portions of the structured assessment object. The provenance data can be stored as field-level provenance, object-level provenance, batch-level provenance, model-level provenance, or tool-call provenance depending on the desired granularity.

The process 500 can improve computing-resource utilization by using content hashes to reuse cached model responses, by performing deterministic numerical checks with verification programs rather than repeated free-form model calls, by using sentence-boundary local reference verification to reduce context-window size and network transmission, and by selecting models through a model registry based on declared capabilities. These operations can reduce duplicate requests, accelerator cycles, memory use, bandwidth use, and retry loops while maintaining a validation trail that can be used for audit, revalidation, and model updating.

The process 500 can also reduce network bandwidth by caching retrieved references and can reduce accelerator utilization by skipping validator calls for locked approved content. The process 500 can also reduce exposure of sensitive patient information by using simulated patient records as training data 375, validation data, or prompt context instead of transmitting source patient records to each generation, validation, or update component. By predicting which candidate model configurations are likely to improve a processing characteristic before those configurations are widely deployed, the process 500 can reduce unnecessary model-training runs, validator calls, inference calls, and rollback operations. The process 500 can provide a technical improvement by allowing proactive actions to be executed during an active assessment session based on streaming response data rather than waiting for completion of the assessment. For example, the assessment generation and validation system can process responses, update a running score, generate replacement assessment objects, validate replacement assessment objects, update access-control records, and schedule external follow-up events faster than a human operator could perform those operations during the same live session. The proactive actions can be executed across multiple users concurrently by queueing generation tasks, canceling stale tasks, validating unpresented replacement objects, and updating interface state without interrupting already-presented assessment objects. By limiting proactive regeneration to unpresented or unlocked content, the process 500 can reduce wasted model calls, reduce inconsistent interface states, and reduce repeated assessment-session restarts. By using tool command(s) 355 to perform proactive scheduling, access-control updates, messaging, and content activation or deactivation, the process 500 can cause technical state changes in external systems based on validated assessment-session data.

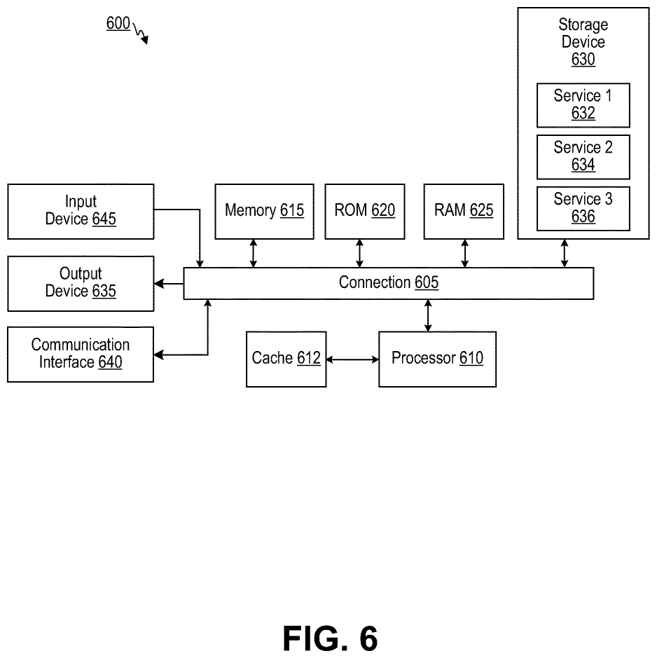

FIG. 6 is a diagram illustrating an example of a system for implementing certain aspects of the present technology. In particular, FIG. 6 illustrates an example of computing system 600, which can be, for example, any computing device making up an internal computing system, a remote computing system, a camera, or any component thereof in which the components of the system are in communication with each other using connection 605. Connection 605 can be a physical connection using a bus, or a direct connection into processor 610, such as in a chipset architecture. Connection 605 can also be a virtual connection, networked connection, or logical connection.

The computing system 600 can include connection 605, processor 610, cache 612, memory 615, read-only memory (ROM) 620, random access memory (RAM) 625, storage device 630, service 1 632, service 2 634, service 3 636, output device 635, communication interface 640, and input device 645. Service 1 632, service 2 634, and service 3 636 can represent executable services, server processes, model-runtime services, retrieval services, verification services, scoring services, interface services, tool-interface services, feedback services, or other software or hardware services. The computing system 600 can implement any of the systems, engines, models, data stores, interfaces, and processes described herein, including the system 100, system 200, AI system 300, interactive assessment interface 400, and process 500.

The processor 610 can include or communicate with hardware accelerators configured for inference, retrieval, ranking, media generation, local verification, encryption, compression, and vector search. Such accelerators can include GPUs, TPUs, NPUs, FPGAs, ASICs, digital signal processors, secure enclaves, cryptographic accelerators, image-signal processors, video encoders, audio processors, and direct-memory-access engines. The cache 612, RAM 625, accelerator memory, and storage device 630 can store model weights, quantized weights, activation caches, key-value caches, vector indexes, embedding tables, prompt caches, response caches, batch queues, cancellation flags, lock records, and audit records used by the assessment generation and validation system. In some examples, accelerator memory stores active key-value caches, embedding vectors, retrieved reference chunks, schema masks, or batch-state tensors while the processor 610 coordinates storage and network operations.

The storage device 630 can store executable services for model orchestration, provider selection, request hashing, request deduplication, response caching, generation-status tracking, database-health monitoring, reference fetching, sentence-aware sectioning, local-model verification, annotation deduplication, formula verification, image compression, media rendering, access control, and audit logging. A database-health service can periodically test a database connection during long-running generation sessions and can reconnect or retry after a connection error, thereby reducing failures during multi-step validation loops. The executable services can be implemented as microservices, local services, containerized services, serverless functions, background workers, scheduled jobs, or hardware-assisted services.

The input device 645 and output device 635 can include medical-simulation hardware, augmented-reality/virtual-reality (AR/VR) hardware, haptic hardware, wearable sensors, implantable sensors, implantable or surface leads, external electrodes, EEG sensors, electromagnetic-field sensors, cameras, microphones, speakers, displays, haptic actuators, and stimulation interfaces. In examples that use stimulation or neuromodulation hardware, the computing system 600 can store permission data, safety limits, device identifiers, stimulation parameters, audit logs, and feedback records, and can coordinate such hardware through sandboxed device drivers or authenticated device APIs. The input device 645 and output device 635 can also include brain-computer-interface probes, neural-interface leads, or other authorized neural sensors or stimulation devices configured to receive learning-state signals or provide controlled stimulation signals. Neural-interface leads, electrodes, and electromagnetic-field sensors can be used as examples of input devices, output devices, or combined input-output devices depending on whether they receive signals, provide stimulation, or both.

The computing system 600 can be a distributed system in which the functions described in this disclosure can be distributed within one or more data centers or a peer network. The described system components can represent multiple instances of such components, each performing some or all of the function for which the component is described. The computing system 600 can include physical devices and/or virtual devices.

In some examples, the computing system 600 implements at least a portion of the AI system 300. For instance, the processor 610 can execute instructions stored in the system memory 615 and/or storage device 630 to implement the ML engine 320, ML model(s) 325, feedback engine(s) 360, and/or other component(s) of the AI system 300. The storage device 630 and/or system memory 615 can store model weights, parameters, hyperparameters, prompts, embeddings, vector indexes, training data 375, data store(s) 380, checkpoint(s), feedback record(s), tool schema(s), tool command history, and/or software instructions for inference, training, fine-tuning, retrieval, tool-command generation, tool invocation, feedback processing, and/or model updating.

In some examples, the AI system 300 is distributed across multiple computing system(s) 600, such that different computing systems host or execute the ML engine 320, ML model(s) 325, feedback engine(s) 360, data store(s) 380, and/or tool(s) 385. The communication interface 640 can transmit and receive input(s) 305, output(s) 330, tool command(s) 355, feedback 365, training data 375, and/or other data among such computing systems. The cache 612, RAM 625, accelerator memory, and/or other high-speed memory can store active model weights, activations, retrieved context, tool responses, and/or batch data while the processor 610 and/or accelerator(s) perform operations of the AI system 300.

The computing system 600 can include at least one processor 610 and connection 605 that couples system memory 615, such as read-only memory (ROM 620) and random access memory (RAM 625), to the processor 610. The computing system 600 can include a cache 612 of high-speed memory connected directly with, in close proximity to, or integrated as part of processor 610 (e.g., registers, on-chip memory).

Processor 610 can include a processor paired with a hardware service or software service, such as services 632, 634, and 636 stored in storage device 630, configured to control processor 610, and/or a special-purpose processor where instructions are incorporated into the actual processor design. Processor 610 may be a self-contained computing system, including multiple cores or processors, a bus, memory controller, and/or cache. A multi-core processor may be symmetric or asymmetric.

To provide for user interaction, computing system 600 includes an input device 645, which can represent any number of input mechanisms, such as a microphone for speech, a touch-sensitive screen for gesture or graphical input, a keyboard, a mouse, and/or a motion detection sensor. Computing system 600 can also include output device 635, which can include a display, a speaker, and/or a haptic feedback actuator. Multimodal systems can allow for provision of multiple types of input/output to communicate with computing system 600. Computing system 600 can include communication interface 640, which can generally govern and manage the user input and system output. The communication interface may perform or facilitate receipt and/or transmission wired or wireless communications using wired and/or wireless transceivers, including those making use of an audio jack/plug, a microphone jack/plug, a universal serial bus (USB) port/plug, an Ethernet port/plug, a fiber optic port/plug, a proprietary wired port/plug, a BLUETOOTH® wireless signal transfer, a BLUETOOTH® low energy (BLE) wireless signal transfer, a radio-frequency identification (RFID) wireless signal transfer, near-field communications (NFC) wireless signal transfer, dedicated short range communication (DSRC) wireless signal transfer, 802.11 Wi-Fi wireless signal transfer, wireless local area network (WLAN) signal transfer, Visible Light Communication (VLC), Worldwide Interoperability for Microwave Access (WiMAX), infrared (IR) communication wireless signal transfer, Public Switched Telephone Network (PSTN) signal transfer, Integrated Services Digital Network (ISDN) signal transfer, third-generation (3G), fourth-generation (4G), Long-Term Evolution (LTE), fifth-generation (5G), and/or sixth-generation (6G) cellular data network wireless signal transfer, ad-hoc network signal transfer, radio wave signal transfer, microwave signal transfer, infrared signal transfer, visible light signal transfer, ultraviolet light signal transfer, and/or wireless signal transfer along the electromagnetic spectrum. The communication interface 640 may include Global Navigation Satellite System (GNSS) receiver(s) or transceiver(s) that are used to determine a location of the computing system 600 based on receipt of signal(s) from satellite(s) associated with GNSS system(s), such as the Global Positioning System (GPS), the Global Navigation Satellite System (GLONASS), the BeiDou Navigation Satellite System (BDS), and Galileo.

The storage device 630 can include instructions (e.g., code) defining software services, servers, model runtime services, inference services, retrieval services, tool interface services, feedback services, update services, and/or other services executable by the processor 610 to cause the system to perform a function. In some examples, a hardware service that performs a particular function can include code stored in a computer-readable medium in connection with the processor 610, connection 605, and/or output device 635.

Claim language or other language reciting “at least one of” a set and/or “one or more” of a set indicates that one member of the set or multiple members of the set (in any combination) satisfy the claim. For example, claim language reciting “at least one of A and B” means A, B, or A and B. In another example, claim language reciting “at least one of A, B, and C” means A, B, C, or A and B, or A and C, or B and C, or A and B and C. The language “at least one of” a set and/or “one or more” of a set does not limit the set to the items listed in the set. For example, claim language reciting “at least one of A and B” can mean A, B, or A and B, and can additionally include items not listed in the set of A and B.

The various illustrative logical blocks, subsystems, circuits, and algorithm steps described in connection with the embodiments disclosed herein may be implemented as electronic hardware, computer software, firmware, or combinations thereof. To clearly illustrate this interchangeability of hardware and software, various illustrative components, blocks, subsystems, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may be implemented in electronic hardware, computer software, firmware, or any combination thereof. Such techniques may be implemented in servers, edge devices, wireless communication device handsets, accelerator-equipped computing devices, integrated circuit devices, medical-simulation devices, or distributed computing systems. Any features described as subsystems or components may be implemented together in an integrated logic device or separately as discrete but interoperable logic devices. If implemented in software, the techniques may be realized at least in part by a non-transitory computer-readable data storage medium comprising program code including instructions that, when executed, perform one or more of the methods described above. The computer-readable medium may comprise memory or data storage media, such as RAM, SDRAM, ROM, NVRAM, EEPROM, flash memory, magnetic data storage media, optical data storage media, solid-state storage, or another non-transitory storage medium.

The program code may be executed by one or more processors, such as one or more digital signal processors (DSPs), microprocessors, application-specific integrated circuits (ASICs), field-programmable gate arrays (FPGAs), graphics processing units (GPUs), tensor processing units (TPUs), neural processing units (NPUs), secure processors, microcontrollers, state machines, or other integrated or discrete logic circuitry. Such processors may be configured to perform any of the techniques described in this disclosure. A processor may also be implemented as a combination of computing devices, e.g., a combination of a DSP and a microprocessor, a plurality of microprocessors, one or more microprocessors in conjunction with an accelerator, or any other suitable configuration. The processors can execute different roles in parallel, such as a first accelerator executing a generation model, a second accelerator executing a validator model, and a central processor coordinating data-store writes and interface updates.

Illustrative aspects of the disclosure include:

Aspect 1. A computer-implemented method for multi-model assessment generation and validation, the method comprising: aggregating assessment parameters and, when available, assessment parameters to generate an enriched prompt; processing the enriched prompt with a generation model to generate or refine a structured assessment object that includes an assessment content portion and a plurality of response-option objects, wherein the plurality of response-option objects include a correct response-option object and at least one incorrect response-option object; auditing the structured assessment object with a plurality of independent validator models; regenerating at least a portion of the structured assessment object based on receipt of a rejection from at least one of the plurality of independent validator models to generate an updated structured assessment object; and generating a score or status based on a plurality of inputs received through an interactive assessment interface, wherein the interactive assessment interface outputs the structured assessment object, and wherein one of the plurality of inputs selects at least one of the plurality of response-option objects.

Aspect 2. The computer-implemented method of Aspect 1, wherein the structured assessment object is a JavaScript Object Notation object.

Aspect 3. The computer-implemented method of any of Aspects 1 to 2, wherein the assessment content portion comprises a question stem.

Aspect 4. The computer-implemented method of any of Aspects 1 to 3, wherein the assessment content portion comprises a clinical vignette.

Aspect 5. The computer-implemented method of any of Aspects 1 to 4, wherein the performance data comprises student performance metrics.

Aspect 6. The computer-implemented method of any of Aspects 1 to 5, wherein the assessment parameters comprise clinical parameters including at least one of a patient age, a patient sex, one or more vital signs, a medical history, a category, a difficulty, a question type, or a case type.

Aspect 7. The computer-implemented method of any of Aspects 1 to 6, wherein the difficulty level controls a similarity between the correct response-option object and the at least one incorrect response-option object.

Aspect 8. The computer-implemented method of any of Aspects 1 to 7, wherein the plurality of independent validator models includes at least one of: models from different model providers or different models from a shared model provider.

Aspect 9. The computer-implemented method of any of Aspects 1 to 8, wherein auditing the structured assessment object comprises medical fact-checking, mathematical verification, temporal verification, logical verification, citation verification, or combinations thereof.

Aspect 10. The computer-implemented method of any of Aspects 1 to 9, wherein the plurality of independent validator models are restricted to comment-only permissions with respect to the structured assessment object.

Aspect 11. The computer-implemented method of any of Aspects 1 to 10, further comprising repeating the auditing and regenerating until an approval condition is satisfied.

Aspect 12. The computer-implemented method of Aspect 11, wherein the approval condition is based on at least one of: approval statuses from the plurality of independent validator models, a quality metric exceeding a quality threshold, a validator-approval count, answer-choice length uniformity, semantic distance among response-option objects, numerical-value distance among response-option objects, or reference-source criteria.

Aspect 13. The computer-implemented method of any of Aspects 1 to 12, further comprising assigning randomized user-facing response labels to the response-option objects.

Aspect 14. The computer-implemented method of any of Aspects 1 to 13, further comprising storing a mapping between a user identifier, an assessment identifier, and a correct response label.

Aspect 15. The computer-implemented method of any of Aspects 1 to 14, further comprising executing a deterministic verification program to verify a numerical value contained in the structured assessment object.

Aspect 16. The computer-implemented method of Aspect 15, wherein the numerical value includes at least one of a body mass index (BMI), a medication dose, an estimated glomerular filtration rate (eGFR), a pressure, a weight, a volume, a mass, or an age.

Aspect 17. The computer-implemented method of any of Aspects 1 to 16, further comprising storing, in a reference tracker, a verification status, a validator model identity, and a reference-source criterion for a citation associated with the structured assessment object, wherein the reference-source criterion includes at least one of a publication-date criterion, a geographic-source criterion, a peer-review criterion, a guideline-source criterion, an open-access criterion, or a paywall criterion.

Aspect 18. The computer-implemented method of any of Aspects 1 to 17, further comprising verifying a reference associated with the structured assessment object by splitting fetched reference content into sentence-boundary sections and processing at least one of the sentence-boundary sections with a local verification model executing on local hardware.

Aspect 19. The computer-implemented method of any of Aspects 1 to 18, further comprising: associating a generation-session identifier with the structured assessment object; and manipulating a model output that is associated with a canceled or superseded generation-session identifier, wherein manipulating the model output includes at least one of discarding the model output or storing the model output as negative training data for further training.

Aspect 20. The computer-implemented method of any of Aspects 1 to 19, further comprising generating a content hash for a model request and reusing a cached model response when the content hash matches a previously stored content hash within a configurable time window.

Aspect 21. The computer-implemented method of any of Aspects 1 to 20, further comprising selecting, using a model registry, a model for a pipeline role based on a declared capability of the model.

Aspect 22. The computer-implemented method of any of Aspects 1 to 21, wherein the interactive assessment interface outputs at least one of audio content, video content, three-dimensional content, augmented-reality content, virtual-reality content, a flashcard, a poll, or a simulation.

Aspect 23. The computer-implemented method of any of Aspects 1 to 22, further comprising receiving sensor data through the interactive assessment interface and adjusting a difficulty, pacing, media modality, or next assessment object based on the sensor data.

Aspect 24. The computer-implemented method of any of Aspects 1 to 23, further comprising generating a batch of structured assessment objects according to a batch context dictionary that specifies category quotas, question-type definitions, case-type definitions, and difficulty-specific requirements.

Aspect 25. The computer-implemented method of any of Aspects 1 to 24, further comprising storing an annotation identifier for a citation associated with the structured assessment object, wherein the annotation identifier is based at least in part on a content hash.

Aspect 26. The computer-implemented method of any of Aspects 1 to 25, further comprising storing cancellation state in a generation-status store and checking the cancellation state before scheduling a model call, tool call, or local verification operation.

Aspect 27. The computer-implemented method of any of Aspects 1 to 26, wherein the at least one incorrect response-option object is based on a previously-generated correct response-option object corresponding to a second assessment content portion of a second structured assessment object.

Aspect 28. The computer-implemented method of any of Aspects 1 to 27, wherein the score indicates that the one of the plurality of inputs selects the correct response-option object corresponding to the assessment content portion.

Aspect 29. The computer-implemented method of any of Aspects 1 to 28, wherein the score indicates that the one of the plurality of inputs selects one of the at least one incorrect response-option object corresponding to the assessment content portion.

Aspect 30. A system for multi-model assessment generation and validation, the system comprising: a memory storing instructions; and a processor that executes the instructions, wherein execution of the instructions by the processor causes the processor to: perform operations according to any of Aspects 1 to 29.

Aspect 31. A non-transitory computer-readable medium having stored thereon instructions that, when executed by at least one processor, cause the at least one processor to perform operations according to any of Aspects 1 to 29.

Aspect 32. An apparatus for multi-model assessment generation and validation, comprising one or more processors, memory, a model registry, a reference tracker, and one or more interfaces configured to perform operations according to any of Aspects 1 to 31.

What is claimed is:

1. A computer-implemented method for multi-model assessment generation and validation, the method comprising:
aggregating assessment parameters to generate an enriched prompt;
processing the enriched prompt with a generation model to generate a structured assessment object that includes an assessment content portion and a plurality of response-option objects, wherein the plurality of response-option objects include a correct response-option object and at least one incorrect response-option object;
auditing the structured assessment object with a plurality of independent validator models;
regenerating at least a portion of the structured assessment object based on receipt of a rejection from at least one of the plurality of independent validator models to generate an updated structured assessment object; and
generating a score based on a plurality of inputs received through an interactive assessment interface, wherein the interactive assessment interface outputs the structured assessment object, and wherein one of the plurality of inputs selects at least one of the plurality of response-option objects.

2. The computer-implemented method of claim 1, wherein the structured assessment object is a JavaScript Object Notation object.

3. The computer-implemented method of claim 1, wherein the assessment content portion comprises a question stem.

4. The computer-implemented method of claim 1, wherein the assessment content portion comprises a clinical vignette.

5. The computer-implemented method of claim 1, wherein the performance data comprises student performance metrics.

6. The computer-implemented method of claim 1, wherein the assessment parameters comprise clinical parameters including at least one of a patient age, a patient sex, one or more vital signs, a medical history, a category, a difficulty, a question type, or a case type.

7. The computer-implemented method of claim 1, wherein the difficulty level controls a similarity between the correct response-option object and the at least one incorrect response-option object.

8. The computer-implemented method of claim 1, wherein the plurality of independent validator models includes at least one of: models from different model providers or different models from a shared model provider.

9. The computer-implemented method of claim 1, wherein auditing the structured assessment object comprises medical fact-checking, mathematical verification, temporal verification, logical verification, citation verification, or combinations thereof.

10. The computer-implemented method of claim 1, wherein the plurality of independent validator models are restricted to comment-only permissions with respect to the structured assessment object.

11. The computer-implemented method of claim 1, further comprising repeating the auditing and regenerating until an approval condition is satisfied.

12. The computer-implemented method of claim 11, wherein the approval condition is based on at least one of: approval statuses from the plurality of independent validator models, a quality metric exceeding a quality threshold, a validator-approval count, answer-choice length uniformity, semantic distance among response-option objects, numerical-value distance among response-option objects, or reference-source criteria.

13. The computer-implemented method of claim 1, further comprising assigning randomized user-facing response labels to the response-option objects.

14. The computer-implemented method of claim 1, further comprising storing a mapping between a user identifier, an assessment identifier, and a correct response label.

15. The computer-implemented method of claim 1, further comprising executing a deterministic verification program to verify a numerical value contained in the structured assessment object.

16. The computer-implemented method of claim 15, wherein the numerical value includes at least one of a body mass index (BMI), a medication dose, an estimated glomerular filtration rate (eGFR), a pressure, a weight, a volume, a mass, or an age.

17. The computer-implemented method of claim 1, further comprising storing, in a reference tracker, a verification status, a validator model identity, and a reference-source criterion for a citation associated with the structured assessment object, wherein the reference-source criterion includes at least one of a publication-date criterion, a geographic-source criterion, a peer-review criterion, a guideline-source criterion, an open-access criterion, or a paywall criterion.

18. The computer-implemented method of claim 1, further comprising verifying a reference associated with the structured assessment object by splitting fetched reference content into sentence-boundary sections and processing at least one of the sentence-boundary sections with a local verification model executing on local hardware.

19. The computer-implemented method of claim 1, further comprising:
associating a generation-session identifier with the structured assessment object; and
manipulating a model output that is associated with a canceled or superseded generation-session identifier, wherein manipulating the model output includes at least one of discarding the model output or storing the model output as negative training data for further training.

20. The computer-implemented method of claim 1, further comprising generating a content hash for a model request and reusing a cached model response when the content hash matches a previously stored content hash within a configurable time window.

21. The computer-implemented method of claim 1, further comprising selecting, using a model registry, a model for a pipeline role based on a declared capability of the model.

22. The computer-implemented method of claim 1, wherein the interactive assessment interface outputs at least one of audio content, video content, three-dimensional content, augmented-reality content, virtual-reality content, a flashcard, a poll, or a simulation.

23. The computer-implemented method of claim 1, further comprising receiving sensor data through the interactive assessment interface and adjusting a difficulty, pacing, media modality, or next assessment object based on the sensor data.

24. The computer-implemented method of claim 1, further comprising generating a batch of structured assessment objects according to a batch context dictionary that specifies category quotas, question-type definitions, case-type definitions, and difficulty-specific requirements.

25. The computer-implemented method of claim 1, further comprising storing an annotation identifier for a citation associated with the structured assessment object, wherein the annotation identifier is based at least in part on a content hash.

26. The computer-implemented method of claim 1, further comprising storing cancellation state in a generation-status store and checking the cancellation state before scheduling a model call, tool call, or local verification operation.

27. The computer-implemented method of claim 1, wherein the at least one incorrect response-option object is based on a previously-generated correct response-option object corresponding to a second assessment content portion of a second structured assessment object.

28. The computer-implemented method of claim 1, wherein the score indicates that the one of the plurality of inputs selects the correct response-option object corresponding to the assessment content portion.

29. The computer-implemented method of claim 1, wherein the score indicates that the one of the plurality of inputs selects one of the at least one incorrect response-option object corresponding to the assessment content portion.

30. A system for multi-model assessment generation and validation, the system comprising:
a memory storing instructions; and
a processor that executes the instructions, wherein execution of the instructions by the processor causes the processor to:
aggregate assessment parameters to generate an enriched prompt;
process the enriched prompt with a generation model to generate a structured assessment object that includes an assessment content portion and a plurality of response-option objects, wherein the plurality of response-option objects include a correct response-option object and at least one incorrect response-option object;
audit the structured assessment object with a plurality of independent validator models;
regenerate at least a portion of the structured assessment object based on receipt of a rejection from at least one of the plurality of independent validator models to generate an updated structured assessment object; and
generate a score based on a plurality of inputs received through an interactive assessment interface, wherein the interactive assessment interface outputs the structured assessment object, and wherein one of the plurality of inputs selects at least one of the plurality of response-option objects.

31. A non-transitory computer-readable storage medium having embodied thereon a program, wherein the program is executable by a processor to perform a method comprising:
aggregating assessment parameters to generate an enriched prompt;
processing the enriched prompt with a generation model to generate a structured assessment object that includes an assessment content portion and a plurality of response-option objects, wherein the plurality of response-option objects include a correct response-option object and at least one incorrect response-option object;
auditing the structured assessment object with a plurality of independent validator models;
regenerating at least a portion of the structured assessment object based on receipt of a rejection from at least one of the plurality of independent validator models to generate an updated structured assessment object; and
generating a score based on a plurality of inputs received through an interactive assessment interface, wherein the interactive assessment interface outputs the structured assessment object, and wherein one of the plurality of inputs selects at least one of the plurality of response-option objects.
